SEQUENCE LISTING (22 sequences)

SEQ ID NO: 1 - RNA, 14 nt, Artificial Sequence
  Synthetic oligonucleotide
cgauacgacagaac

SEQ ID NO: 2 - RNA, 5 nt, Artificial Sequence
  Synthetic oligonucleotide
cgccg

SEQ ID NO: 3 - RNA, 7 nt, Artificial Sequence
  Synthetic oligonucleotide
cggccgc

SEQ ID NO: 4 - RNA, 5 nt, Artificial Sequence
  Synthetic oligonucleotide
cgcgc

SEQ ID NO: 5 - RNA, 4 nt, Artificial Sequence
  Synthetic oligonucleotide
cgau

SEQ ID NO: 6 - RNA, 38 nt, Artificial Sequence
  Synthetic oligonucleotide
  misc_feature (1)..(3): nucleotide with 2′OMe modification
  misc_feature (1)..(3): nucleotide with a 3′ phosphorothioate modification
aucgacaagaaagggacugaguuuuagagcuaugcugu

SEQ ID NO: 7 - RNA, 82 nt, Artificial Sequence
  Synthetic oligonucleotide
  misc_feature (79)..(81): nucleotide with 2′OMe modification
  misc_feature (79)..(81): nucleotide with a 3′ phosphorothioate modification
cuuggaaacaagacagcauagcaaguuaaaauaaggcuaguccguuaucaacuugaaaaaguggcaccgagucggugcuuuu

SEQ ID NO: 8 - RNA, 47 nt, Artificial Sequence
  Synthetic oligonucleotide
  misc_feature (1)..(3): nucleotide with 2′OMe modification
  misc_feature (1)..(3): nucleotide with a 3′ phosphorothioate modification
aucgacaagaaagggacugaguuuuagagcuaugcugucuugcucga

SEQ ID NO: 9 - RNA, 76 nt, Artificial Sequence
  Synthetic oligonucleotide
  misc_feature (73)..(75): nucleotide with 2′OMe modification
  misc_feature (73)..(75): nucleotide with a 3′ phosphorothioate modification
uacaagacagcauagcaaguuaaaauaaggcuaguccguuaucaacuugaaaaaguggcaccgagucggugcuuuu

SEQ ID NO: 10 - RNA, 31 nt, Artificial Sequence
  Synthetic oligonucleotide
  misc_feature (1)..(3): nucleotide with 2′OMe modification
  misc_feature (1)..(3): nucleotide with a 3′ phosphorothioate modification
aucgacaagaaagggacugagcccuagagcg

SEQ ID NO: 11 - RNA, 71 nt, Artificial Sequence
  Synthetic oligonucleotide
  misc_feature (68)..(70): nucleotide with 2′OMe modification
  misc_feature (68)..(70): nucleotide with a 3′ phosphorothioate modification
gcgaaagccgcaaguuaggguaaggcuaguccguuaucaacuugaaaaagugggaccgagucggugcuuuu

SEQ ID NO: 12 - RNA, 32 nt, Artificial Sequence
  Synthetic oligonucleotide
  misc_feature (1)..(3): nucleotide with 2′OMe modification
  misc_feature (1)..(3): nucleotide with a 3′ phosphorothioate modification
aucgacaagaaagggacugagcccuagagccg

SEQ ID NO: 13 - RNA, 74 nt, Artificial Sequence
  Synthetic oligonucleotide
  misc_feature (71)..(73): nucleotide with 2′OMe modification
  misc_feature (71)..(73): nucleotide with a 3′ phosphorothioate modification
gcggaaacgccggcaaguuaggguaaggcuaguccguuaucaacuugaaaaagugggaccgagucggugcuuuu

SEQ ID NO: 14 - RNA, 32 nt, Artificial Sequence
  Synthetic oligonucleotide
  misc_feature (1)..(3): nucleotide with 2′OMe modification
  misc_feature (1)..(3): nucleotide with a 3′ phosphorothioate modification
aucgacaagaaagggacugaguuuuagagccg

SEQ ID NO: 15 - RNA, 74 nt, Artificial Sequence
  Synthetic oligonucleotide
  misc_feature (71)..(73): nucleotide with 2′OMe modification
  misc_feature (71)..(73): nucleotide with a 3′ phosphorothioate modification
gcggaaacgccggcaaguuaaaauaaggcuaguccguuaucaacuugaaaaagugggaccgagucggugcuuuu

SEQ ID NO: 16 - RNA, 30 nt, Artificial Sequence
  Synthetic oligonucleotide
  misc_feature (1)..(3): nucleotide with 2′OMe modification
  misc_feature (1)..(3): nucleotide with a 3′ phosphorothioate modification
aucgacaagaaagggacugaguuuuagagc

SEQ ID NO: 17 - RNA, 72 nt, Artificial Sequence
  Synthetic oligonucleotide
  misc_feature (69)..(71): nucleotide with 2′OMe modification
  misc_feature (69)..(71): nucleotide with a 3′ phosphorothioate modification
gcggaaacgcgcaaguuaaaauaaggcuaguccguuaucaacuugaaaaagugggaccgagucggugcuuuu

SEQ ID NO: 18 - RNA, 30 nt, Artificial Sequence; identical to SEQ ID NO: 16
  Synthetic oligonucleotide
  misc_feature (1)..(3): nucleotide with 2′OMe modification
  misc_feature (1)..(3): nucleotide with a 3′ phosphorothioate modification

SEQ ID NO: 19 - RNA, 70 nt, Artificial Sequence
  Synthetic oligonucleotide
  misc_feature (67)..(69): nucleotide with 2′OMe modification
  misc_feature (67)..(69): nucleotide with a 3′ phosphorothioate modification
uagaaauagcaaguuaaaauaaggcuaguccguuaucaacuugaaaaagugggaccgagucggugcuuuu

SEQ ID NO: 20 - RNA, 70 nt, Artificial Sequence; identical to SEQ ID NO: 19
  Synthetic oligonucleotide
  misc_feature (1)..(3): nucleotide with 2′OMe modification
  misc_feature (29)..(30): nucleotide with a 3′ phosphorothioate modification

SEQ ID NO: 21 - RNA, 72 nt, Artificial Sequence
  Synthetic oligonucleotide
  misc_feature (1)..(12): nucleotide with 2′OMe modification
  misc_feature (41)..(72): nucleotide with 2′OMe modification
  misc_feature (69)..(71): nucleotide with a 3′ phosphorothioate modification
gcggaaacgcgcaaguuaaaauaaggcuaguccguuaucaacuugaaaaaguggcaccgagucggugcuuuu

SEQ ID NO: 22 - RNA, 32 nt, Artificial Sequence
  Synthetic oligonucleotide
  misc_feature (1)..(3): nucleotide with 2′OMe modification
  misc_feature (1)..(3): nucleotide with a 3′ phosphorothioate modification
gaacacaaagcauagacugcguuuuagagccg

(19) United States
(12) Patent Application Publication
(10) Pub. No.: US 2023/0055682 A1
(43) Pub. Date: Feb. 23, 2023

(54) SYNTHETIC GUIDE RNA, COMPOSITIONS, METHODS, AND USES THEREOF

(71) Applicant: Beam Therapeutics lnc., Cambridge, MA (US)

(72) Inventors: Brian Cafferty, Lexington, MA (US); Aaron Larsen, Cambridge, MA (US)

(21) Appl. No.: 17/782,045

(22) PCT Filed: Dec. 3, 2020
(86) PCT No.: PCT/US20/63084

§ 371 Date: Jun. 2, 2022

Related U.S. Application Data
(60) Provisional application No. 62/943,158, filed on Dec. 3, 2019.
(60) Provisional application No. 63/031,262, filed on May 28, 2020.

(51) Int. Cl. C12N 15/90; C12N 15/11; C12N 9/22

(52) U.S. Cl. CPC C12N 15/907; C12N 15/11; C12N 9/22; C12N 2310/20; C12N 2310/531; C12N 2800/80

(57) ABSTRACT

The present invention provides, among other things, a method of producing a synthetic RNA using a self-templating approach. For example, in some embodiments a synthetic gRNA is produced comprising: contacting a first RNA with a second RNA, wherein the first RNA and the second RNA comprise at least five RNA nucleotides that are complementary, and wherein the contacting forms a stem structure or a stem loop structure, and ligating the first RNA and the second RNA with a ligating enzyme (i) within the stem structure, or (ii) at an end of the stem structure, thereby forming a loop at the end of the stem structure.

### CROSS-REFERENCE TO RELATED APPLICATIONS

This application claims benefit of, and priority to, U.S. 62/943,158 filed on Dec. 3, 2019, and U.S. 63/031,262 filed on May 28, 2020, the contents of each of which are incorporated herein.

### BACKGROUND

Guide RNA molecules (gRNA) in association with Cas endonucleases, and related enzymes—including base editors—are used for applications in gene editing. A common form of gRNA used for therapeutic applications are single, non-natural RNAs of approximately 100 nucleotides that form ribonucleoproteins with Cas9. Plasmid DNA and solid-phase synthesis using phosphoramidite chemistry are typical approaches to obtain therapeutic sgRNAs. Use of synthetic RNA is advantageous as it allows for incorporation of modifications that can both increase the chemical stability of sgRNA and reduce editing of genomic DNA at undesired locations (off-targets).

Problems remain in the manufacture of gRNAs, for example: i) the length of sgRNA molecules, typically 100 nucleotides in length, pushes the limit of phosphoramidite chemistry. Phosphoramidite chemistry has a coupling efficiency for RNA of ˜0.985× (where X is the number of nucleotides). For example, synthesis of a gRNA of 100 nts long yields approximately 20% full length product before isolation. These lengths are significantly greater than those used in oligonucleotide-based therapeutics currently on the market (siRNA and antisense oligonucleotides (ASOs) are typically 20-50 nucleotides in length and thus more amenable to purification); ii) complete removal of side products formed from incomplete coupling (truncation products), incomplete deprotection, and random insertions of nucleotides (addition products) is not currently achievable for RNA at these length scales by standard purification methods (e.g., chromatography, electrophorese); and iii) side-products isolated with the full-length product, which have similar sequence homology to the full-length product, will reduce the activity of the ribonucleoprotein complex and may lead to off-target editing.

### SUMMARY OF THE INVENTION

Described herein is a process to synthesize gRNA using a chemical and/or enzymatic strategy that overcomes challenges that limit the purity, integrity and final (post-purified) yield of synthetic RNAs. The invention provides, in some aspects, a ligation-based approach where two or more synthetic RNAs are ligated using an enzyme. It was surprisingly found that a ligation-based approach increases the purity, yield and the integrity of the gRNA produced. The resultant purity, yield and integrity of the gRNA allows for increased editing efficiencies and a reduction of off-target editing as compared to previous methods of gRNA synthesis. The ligation-based method for the synthesis of gRNA includes methods comprising using two or more partially complementary synthetic RNAs that are subsequently ligated (“template approach”), and methods that do not require complementarity between two or more synthetic RNAs (“non-templated approach”).

In some aspects, a method a method is provided comprising, contacting a first RNA with a second RNA, wherein the first RNA and the second RNA comprise at least five RNA nucleotides that are complementary, and wherein the contacting forms a stem structure or a stem loop structure, and ligating the first RNA and the second RNA with a ligating enzyme (i) within the stem structure, or (ii) at an end of the stem structure, thereby forming a loop at the end of the stem structure.

In some embodiments, the contacting forms a stem structure and the ligating enzyme ligates the first RNA and the second RNA at an end of the stem structure, thereby forming a loop at the end of the stem structure.

In some embodiments, the contacting forms a stem loop structure and the ligating enzyme ligates the first RNA and the second RNA within a stem of the stem loop structure.

In some embodiments, the ligating enzyme is selected from the group consisting of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, SplintR Ligase E. coli DNA Ligase, 9° N DNA Ligase, CircLigase, CircLigase II, DNA Ligase I, DNA Ligase III, and DNA Ligase IV. Accordingly, in some embodiments, the ligating enzyme is T4 RNA ligase 1. In some embodiments, the ligating enzyme is T4 RNA ligase 2. In some embodiments, the ligating enzyme is RtcB Ligase. In some embodiments, the ligating enzyme is Thermo-stable 5′ App DNA/RNA Ligase. In some embodiments, the ligating enzyme is ElectroLigase. In some embodiments, the ligating enzyme is T4 DNA Ligase. In some embodiments, the ligating enzyme is T3 DNA Ligase. In some embodiments, the ligating enzyme is T7 DNA Ligase. In some embodiments, the ligating enzyme is Taq DNA Ligase. In some embodiments, the ligating enzyme is SplintR Ligase E. coli DNA Ligase. In some embodiments, the ligating enzyme is 9° N DNA Ligase. In some embodiments, the ligating enzyme is CircLigase. In some embodiments, the ligating enzyme is CircLigase II. In some embodiments, the ligating enzyme is DNA Ligase I. In some embodiments, the ligating enzyme is DNA Ligase III. In some embodiments, the ligating enzyme is DNA Ligase IV.

In some embodiments, the first and/or second RNA is chemically synthesized.

In some embodiments, the first RNA is a clustered regularly interspersed short palindromic repeats (CRISPR) RNA (crRNA) and the second RNA is a trans-activating RNA (tracrRNA).

In some embodiments, a guide RNA (gRNA) is produced according to the methods herein.

In some embodiments, the first RNA and/or the second RNA is chemically synthesized.

In some embodiments, the first and/or the second RNA is enzymatically synthesized.

In some embodiments, the first RNA and/or the second RNA comprises a modified base. Various modified RNA bases are known in the art and include for example, 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′OMethyl modifications can also be used with the methods described herein. For example, the following RNA comprising one or more of the following 2′OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the first RNA and/or second RNA comprises one or more of the following modifications: phosphorothioates, 2′O-methyls, 2′ fluoro (2′F), DNA.

In some embodiments, the first RNA and/or the second RNA comprises 2′OMe modifications at the 3′ and 5′-ends.

In some embodiments, the first RNA and/or second RNA comprises one or more of the following modifications: 2′-O-2-Methoxyethyl (MOE), locked nucleic acids, bridged nucleic acids, unlocked nucleic acids, peptide nucleic acids, morpholino nucleic acids.

In some embodiments, the first RNA and/or second RNA comprises one or more of the following base modifications: 2,6-diaminopurine, 2-aminopurine, pseudouracil, N1-methyl-psuedouracil, 5′ methyl cytosine, 2′pyrimidinone (zebularine), thymine.

Other modified bases include for example, 2-Aminopurine, 5-Bromo dU, deoxyUridine, 2,6-Diaminopurine (2-Amino-dA), Dideoxy-C, deoxylnosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Desoxy-r(A,C,G,U), 3′-O-Methyl-r(A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the first RNA and/or second RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

In some embodiments, ligating the first RNA and the second RNA with a ligating enzyme creates phosphodiester linkages between the first and the second RNA.

In some embodiments, the first RNA and/or second RNA nucleotide is engineered to allow for non-covalent assembly.

In some embodiments, the stem loop has a length of between about 2-50 nucleotides.

In some embodiments, the first RNA and the second RNA comprise at least two RNA nucleotides that have perfect complementarity.

In some embodiments, the first RNA and the second RNA comprise at least three, four, fix, six or seven consecutive RNA nucleotides that have perfect complementarity.

In some embodiments, the RNA nucleotides that have perfect complementarity are present in a top stem and/or in a bottom stem.

In some embodiments, the first RNA and the second RNA comprise at least five, six, or seven consecutive RNA nucleotides that are complementary at a lower stem formed by the first RNA and the second RNA.

In some embodiments, the first RNA and the second RNA comprise at least four to fourteen consecutive RNA nucleotides that are complementary at an upper stem.

In some embodiments, the first RNA and the second RNA comprise four consecutive RNA nucleotides that are complementary at an upper stem.

In some embodiments, the first and the second RNA comprise five consecutive RNA nucleotides that are complementary at an upper stem.

In some embodiments, the first and the second RNA comprise seven consecutive RNA nucleotides that are complementary at an upper stem.

In some embodiments, the first and the second RNA comprises 14 consecutive RNA nucleotides that are complementary at an upper stem.

In some embodiments, the first and the second RNA comprise 7 consecutive RNA nucleotides that are complementary at a lower stem.

In some embodiments, the first RNA and/or the second RNA is engineered to create a ligation site for a ligation enzyme.

In some embodiments, the stem loop comprises a loop of 4, 5, 6, 7, 8, 9, 10, 11, 12, 13, 14, 15 or 16 nucleotides. Accordingly, in some embodiments, the stem loop comprises a loop of 4 nucleotides, also referred to herein as a tetraloop. In some embodiments, the stem loop comprises a loop of 5 nucleotides. In some embodiments, the stem loop comprises a loop of 6 nucleotides. In some embodiments, the stem loop comprises a loop of 7 nucleotides. In some embodiments, the stem loop comprises a loop of 8 nucleotides. In some embodiments, the stem loop comprises a loop of 9 nucleotides. In some embodiments, the stem loop comprises a loop of 10 nucleotides. In some embodiments, the stem loop comprises a loop of 11 nucleotides. In some embodiments, the stem loop comprises a loop of 12 nucleotides. In some embodiments, the stem loop comprises a loop of 13 nucleotides. In some embodiments, the stem loop comprises a loop of 14 nucleotides. In some embodiments, the stem loop comprises a loop of 15 nucleotides. In some embodiments, the stem loop comprises a loop of 15 nucleotides.

In some embodiments, ligating the first RNA and the second RNA occurs at a ligation site that is at least 1, 2, 3, 4, 5, 6, 7, 8, 9, or 10 base pairs from the loop. Accordingly, in some embodiments, ligating the first RNA and the second RNA occurs at a ligation site that is at least 1 base pair from the loop. In some embodiments, ligating the first RNA and the second RNA occurs at a ligation site that is at least 2 base pairs from the loop. In some embodiments, ligating the first RNA and the second RNA occurs at a ligation site that is at least 3 base pairs from the loop. In some embodiments, ligating the first RNA and the second RNA occurs at a ligation site that is at least 4 base pairs from the loop. In some embodiments, ligating the first RNA and the second RNA occurs at a ligation site that is at least 5 base pairs from the loop. In some embodiments, ligating the first RNA and the second RNA occurs at a ligation site that is at least 6 base pairs from the loop. In some embodiments, ligating the first RNA and the second RNA occurs at a ligation site that is at least 7 base pairs from the loop. In some embodiments, ligating the first RNA and the second RNA occurs at a ligation site that is at least 8 base pairs from the loop. In some embodiments, ligating the first RNA and the second RNA occurs at a ligation site that is at least 9 base pairs from the loop. In some embodiments, ligating the first RNA and the second RNA occurs at a ligation site that is at least 10 base pairs from the loop.

In some embodiments, the ligation site is 2 or 3 base pairs from the loop.

In some embodiments, ligating the first RNA and the second RNA occurs at a ligation site that is at least 3, 4, 5, 6, 7, 8, 9, 10, 11, or 12 base pairs from a bulge. Accordingly, in some embodiments, ligating the first RNA and the second RNA occurs at a ligation site that is at least 3 base pairs from a bulge. In some embodiments, ligating the first RNA and the second RNA occurs at a ligation site that is at least 4 base pairs from a bulge. In some embodiments, ligating the first RNA and the second RNA occurs at a ligation site that is at least 5 base pairs from a bulge. In some embodiments, ligating the first RNA and the second RNA occurs at a ligation site that is at least 6 base pairs from a bulge. In some embodiments, ligating the first RNA and the second RNA occurs at a ligation site that is at least 7 base pairs from a bulge. In some embodiments, ligating the first RNA and the second RNA occurs at a ligation site that is at least 8 base pairs from a bulge. In some embodiments, ligating the first RNA and the second RNA occurs at a ligation site that is at least 9 base pairs from a bulge. In some embodiments, ligating the first RNA and the second RNA occurs at a ligation site that is at least 10 base pairs from a bulge. In some embodiments, ligating the first RNA and the second RNA occurs at a ligation site that is at least 11 base pairs from a bulge. In some embodiments, ligating the first RNA and the second RNA occurs at a ligation site that is at least 12 base pairs from a bulge.

In some embodiments, ligating the first RNA and the second RNA occurs at a ligation site that is 3, 4, 5, or 11 base pairs from the bulge.

In some embodiments, the first RNA and/or second RNA is enzymatically produced.

In some embodiments, the first RNA comprises a 3′ sequence that is capable of base pairing with a portion of the second RNA.

In some embodiments, the first RNA comprises a phosphate at the 5′ terminus.

In some embodiments, the first RNA is a donor RNA.

In some embodiments, the second RNA comprises a variable protospacer region.

In some embodiments, the second RNA is an acceptor RNA.

In some embodiments, the first RNA comprises an adenosine triphosphate at the 5′ terminus.

In some embodiments, about 8-50 nucleotides are complementary and allow for base pairing between the first and the second RNA. In some embodiments, about 8-40 nucleotides are complementary and allow for base pairing between the first and the second RNA. In some embodiments, about 8-30 nucleotides are complementary and allow for base pairing between the first and the second RNA. In some embodiments, about 8-20 nucleotides are complementary and allow for base pairing between the first and the second RNA. In some embodiments, about 8-10 nucleotides are complementary and allow for base pairing between the first and the second RNA

In some embodiments, the 8-50 nucleotides are partially complementary. In some embodiments, about 8-40 nucleotides are partially complementary and allow for base pairing between the first and the second RNA. In some embodiments, about 8-30 nucleotides are partially complementary and allow for base pairing between the first and the second RNA. In some embodiments, about 8-20 nucleotides are partially complementary and allow for base pairing between the first and the second RNA. In some embodiments, about 8-10 nucleotides are partially complementary and allow for base pairing between the first and the second RNA.

In some embodiments, the 8-50 nucleotides are from about 50% to 99% complementary.

In some embodiments, the 8-50 nucleotides are perfectly complementary. In some embodiments, about 8-40 nucleotides are perfectly complementary and allow for base pairing between the first and the second RNA. In some embodiments, about 8-30 nucleotides are perfectly complementary and allow for base pairing between the first and the second RNA. In some embodiments, about 8-20 nucleotides are perfectly complementary and allow for base pairing between the first and the second RNA. In some embodiments, about 8-10 nucleotides are perfectly complementary and allow for base pairing between the first and the second RNA

In some embodiments, the first and the second RNA have different nucleotide lengths.

In some embodiments, the first RNA has from about 20-100 nucleotides. In some embodiments, the first RNA has about 20-90 nucleotides. In some embodiments, the first RNA has about 20-80 nucleotides. In some embodiments, the first RNA has about 20-70 nucleotides. In some embodiments, the first RNA has about 20-60 nucleotides. In some embodiments, the first RNA has about 20-50 nucleotides. In some embodiments, the first RNA has about 20-40 nucleotides. In some embodiments, the first RNA has about 20-30 nucleotides.

In some embodiments, the second RNA has from about 20-70 nucleotides. In some embodiments, the second RNA has about 20-60 nucleotides. In some embodiments, the second RNA has about 20-50 nucleotides. In some embodiments, the second RNA has about 20-40 nucleotides. In some embodiments, the second RNA has about 20-30 nucleotides.

In some embodiments, base pairing occurs in a lower stem.

In some embodiments, 7 nucleotides are complementary in the lower stem and allow for base pairing between the first RNA and the second RNA.

In some embodiments, the base pairing occurs in an upper stem.

In some embodiments, 2 nucleotides are complementary in the upper stem and allow for base pairing between the first RNA and the second RNA.

In some embodiments, the gRNA has a length of about 100 nucleotides, about 125 nucleotides, about 150 nucleotides, about 175 nucleotides, about 200 nucleotides, or greater than about 200 nucleotides. Accordingly, in some embodiments, the gRNA has a length of about 100 nucleotides. In some embodiments, the gRNA has a length of about 125 nucleotides. In some embodiments, the gRNA has a length of about 150 nucleotides. In some embodiments, the gRNA has a length of about 175 nucleotides. In some embodiments, the gRNA has a length of about 200 nucleotides. In some embodiments, the gRNA has a length of greater than 200 nucleotides.

In some embodiments, the gRNA is an extended guide RNA, prime editor guide RNA (pegRNA), or a Cas12 guide RNA such as Cas12a guide RNA, Cas12b guide RNA, Cas12c guide RNA, Cas12d, guide RNA, Cas12e guide RNA, Cas12f guide RNA, Cas12g guide RNA, Cas12h guide RNA, Cas12i guide RNA, Cas12j guide RNA, or Cas12k guide RNA. Accordingly, in some embodiments, the gRNA is an extended guide RNA. In some embodiments, the gRNA is a prime editor guide RNA (pegRNA). In some embodiments, the gRNA is a Cas12 guide RNA. Various Cas12 are known and the art, and include for example Cas12 from Class 2 CRISPR-Cas systems. Exemplary Cas12 include for example, any Cas12 from Class 2 CRISPR-Cas systems. In some embodiments, the methods described herein are suitable to synthesize gRNA for Cas12a, Cas12b, Cas12c, Cas12d, Cas12e, Cas12f, Cas12g, Cas12h, Cas12i, Cas12j, and/or Cas12k. Accordingly, in some embodiments, the gRNA is a Cas12a guide RNA. In some embodiments, the gRNA is a Cas12b guide RNA. In some embodiments, the gRNA is a Cas12c guide RNA. In some embodiments, the gRNA is a Cas12d guide RNA. In some embodiments, the gRNA is a Cas12e guide RNA. In some embodiments, the gRNA is a Cas12f guide RNA. In some embodiments, the gRNA is a Cas12g guide RNA. In some embodiments, the gRNA is a Cas12h guide RNA. In some embodiments, the gRNA is a Cas12i guide RNA. In some embodiments, the gRNA is a Cas12j guide RNA. In some embodiments, the gRNA is a Cas12k guide RNA.

In some embodiments, the gRNA comprises one or more of the following: a spacer, a lower stem, a bulge, an upper stem, a nexus and a hairpin.

In some embodiments, the first RNA and the second RNA are present at a ratio of about 0.5:1, 0.6:1, 0.7:1, 0.8:1, 0.9:1, 1:1, 1:0.9, 1:0.8, 1:0.7, 1:0.6, or 1:0.5. Accordingly, in some embodiments, the first RNA and the second RNA are present at a ratio of about 0.5:1. In some embodiments, the first RNA and the second RNA are present at a ratio of about 0.6:1. In some embodiments, the first RNA and the second RNA are present at a ratio of about 0.7:1. In some embodiments, the first RNA and the second RNA are present at a ratio of about 0.8:1. In some embodiments, the first RNA and the second RNA are present at a ratio of about 0.9:1. In some embodiments, the first RNA and the second RNA are present at a ratio of about 1:1. In some embodiments, the first RNA and the second RNA are present at a ratio of about 1:0.9. In some embodiments, the first RNA and the second RNA are present at a ratio of about 1:0.8. In some embodiments, the first RNA and the second RNA are present at a ratio of about 1:0.7. In some embodiments, the first RNA and the second RNA are present at a ratio of about 1:0.6. In some embodiments, the first RNA and the second RNA are present at a ratio of about 1:0.5.

In some embodiments, the gRNA is produced at a yield of about 50%, 55%, 60%, 65%, 70%, 75%, 80%, 85%, 90%, 95%, 99% or more. Accordingly, in some embodiments, the gRNA is produced at a yield of about 50%. In some embodiments, the gRNA is produced at a yield of about 55%. In some embodiments, the gRNA is produced at a yield of about 60%. In some embodiments, the gRNA is produced at a yield of about 65%. In some embodiments, the gRNA is produced at a yield of about 70%. In some embodiments, the gRNA is produced at a yield of about 75%. In some embodiments, the gRNA is produced at a yield of about 80%. In some embodiments, the gRNA is produced at a yield of about 85%. In some embodiments, the gRNA is produced at a yield of about 90%. In some embodiments, the gRNA is produced at a yield of about 95%. In some embodiments, the gRNA is produced at a yield of more than 99%.

In some embodiments, the gRNA is produced at 50%, 55%, 60%, 65%, 70%, 75%, 80%, 85%, 90%, 95%, 99% or more improvement in yield as compared to conventional synthetic methods. Accordingly, in some embodiments, the gRNA is produced at 50% improvement in yield as compared to conventional synthetic methods. In some embodiments, the gRNA is produced at 55% improvement in yield as compared to conventional synthetic methods. In some embodiments, the gRNA is produced at 60% improvement in yield as compared to conventional synthetic methods. In some embodiments, the gRNA is produced at 55% improvement in yield as compared to conventional synthetic methods. In some embodiments, the gRNA is produced at 60% improvement in yield as compared to conventional synthetic methods. In some embodiments, the gRNA is produced at 65% improvement in yield as compared to conventional synthetic methods. In some embodiments, the gRNA is produced at 70% improvement in yield as compared to conventional synthetic methods. In some embodiments, the gRNA is produced at 75% improvement in yield as compared to conventional synthetic methods. In some embodiments, the gRNA is produced at 80% improvement in yield as compared to conventional synthetic methods. In some embodiments, the gRNA is produced at 85% improvement in yield as compared to conventional synthetic methods. In some embodiments, the gRNA is produced at 90% improvement in yield as compared to conventional synthetic methods. In some embodiments, the gRNA is produced at 99% improvement in yield as compared to conventional synthetic methods. In some embodiments, the gRNA is produced at more than 99% improvement in yield as compared to conventional synthetic methods.

In some aspects, a method of producing a synthetic guide RNA (gRNA) is provided comprising: providing a first RNA comprising a 5′-monophosphate; providing a second RNA; providing an oligonucleotide that has partial complementarity to the first RNA and the second RNA, wherein the complementarity of the oligonucleotide allows for base pairing with the first and the second RNA; and providing a ligase to catalyze ligation between the first and the second RNA, thus producing the synthetic gRNA.

In some aspects, a method of producing a synthetic guide RNA (gRNA) is provided comprising: providing a first RNA comprising a 5′-monophosphate; providing a second RNA comprising a blocked 3′ end; and providing a ligase to catalyze ligation between the first and the second RNA, thus producing the synthetic gRNA.

In some embodiments, the first RNA is a trans-activating RNA (tracrRNA), and the second RNA is a clustered regularly interspersed short palindromic repeats (CRISPR) RNA (crRNA).

In some embodiments, the oligonucleotide is about 100 nucleotides long. In some embodiments, the oligonucleotide is about 80, 90, 100, 110, 120, 130, 140, 150, 160, 170, 180, 190 or 200 nucleotides long.

In some aspects, a method of producing a synthetic guide RNA (gRNA) is provided comprising:

providing two or more RNA fragments; providing an oligonucleotide that has partial complementarity to the two or more RNA fragments, wherein the complementarity of the oligonucleotide allows for base pairing with the two or more RNA fragments; and providing a ligase to catalyze ligation between the two or more RNA fragments, thus producing the synthetic guide RNA.

In some embodiments, the two or more RNA fragments are ligated at an overhang, blunt end, or at a bulge.

In some embodiments, a guide RNA (gRNA), or prime editing guide RNA (pegRNA) is synthesized by a method a method described herein. In some embodiments, the guide RNA is a Cas9 guide RNA or a Cas12 guide RNA.

The methods described herein can be used to synthesize Cas12 guide RNA, such as a Cas12b guide RNA. For example, Cas12b RNA harpin loop structures can be targeted as positions to split the sgRNA. Various hairpin loop structures can be targeted as positions to split the sgRNA, for example, such as those hairpin loop structures as shown in FIG. 16. Accordingly, in some embodiments, the Cas12 guide RNA can be synthesized in accordance with the methods described herein by targeting one or more hairpin loop structures. In some embodiments, one or more tetraloops within Cas12 RNA is targeted for ligation. For example, in some embodiments, one or more tetraloops within Cas12b RNA is target for ligation. In some embodiments, the tetraloop that is targeted is located at the 5′ end of the Cas12 RNA. In some embodiments, the tetraloop that is targeted is located at the 3′ end of the Cas12 RNA. In some embodiments, the targeted tetraloop is located within about 5-30 nucleotides from the 3′ end of the Cas12 RNA. In some embodiments, the targeted tetraloop is about 5-30 nucleotides from the 5′ end of the Cas12 RNA.

In some aspects, a method for targeted transcription activation, targeted transcription repression, targeted epigenome modification, or targeted genome modification is provided, the method comprising introducing into a eukaryotic cell: (a) a synthetic guide RNA (gRNA) as defined in any one of the preceding claims; (b) at least one CRISPR/Cas protein or a nucleic acid encoding the at least one CRISPR/Cas protein; wherein interactions between (a) and (b) and a target sequence in chromosomal DNA leads to targeted transcription activation, targeted transcription repression, targeted epigenome modification, or targeted genome modification.

In some aspects, a method for targeted RNA modification is provided, the method comprising introducing into a eukaryotic cell: (a) a synthetic guide RNA (gRNA) as defined in any one of the preceding claims; (b) at least one CRISPR/Cas protein or a nucleic acid encoding the at least one CRISPR/Cas protein; wherein interactions between (a) and (b) and an RNA expressed by chromosomal DNA leads to a modification of the RNA expressed by the chromosomal DNA.

In some embodiments, the RNA expressed by the chromosomal DNA is a messenger RNA (mRNA).

In some embodiments, the CRISPR/Cas protein is selected from Cas9, Cpf1, SaCas, Cas12, Cas13, or modified versions thereof.

In some embodiments, a method for producing synthetic guide RNA (gRNA) is provided according methods described herein.

In some embodiments, the second RNA comprises a 3′ sequence that is capable of base pairing with a portion of the first RNA.

In some embodiments, the second RNA comprises a variable protospacer region.

In some embodiments, the first RNA comprises a phosphate at the 5′ terminus.

In some embodiments, the contacting forms a stem loop structure and the ligating enzyme ligates the first RNA and the second RNA within a stem of the stem loop structure.

In some embodiments, the ligating enzyme is T4 RNA ligase 2.

In some embodiments, the stem loop comprises GC base pairs in the upper stem.

In some embodiments, the upper stem comprises a nucleotide sequence at least about 80% identical to CGAUACGACAGAAC. In some embodiments, the upper stem comprises a nucleotide sequence at least about 85% identical to CGAUACGACAGAAC. In some embodiments, the upper stem comprises a nucleotide sequence at least about 90% identical to CGAUACGACAGAAC. In some embodiments, the upper stem comprises a nucleotide sequence at least about 95% identical to CGAUACGACAGAAC. In some embodiments, the upper stem comprises a nucleotide sequence at least about 99% identical to CGAUACGACAGAAC. In some embodiments, the upper stem comprises a nucleotide sequence is identical to CGAUACGACAGAAC.

In some embodiments, the upper stem comprises a nucleotide sequence at least about 80% identical to CGCCG. In some embodiments, the upper stem comprises a nucleotide sequence at least about 85% identical to CGCCG. In some embodiments, the upper stem comprises a nucleotide sequence at least about 90% identical to CGCCG. In some embodiments, the upper stem comprises a nucleotide sequence at least about 80% identical to CGCCG. In some embodiments, the upper stem comprises a nucleotide sequence at least about 95% identical to CGCCG. In some embodiments, the upper stem comprises a nucleotide sequence at least about 99% identical to CGCCG. In some embodiments, the upper stem comprises a nucleotide sequence is identical to CGCCG.

In some embodiments, the upper stem comprises a nucleotide sequence at least about 80% identical to CGGCCGC. In some embodiments, the upper stem comprises a nucleotide sequence at least about 85% identical to CGGCCGC. In some embodiments, the upper stem comprises a nucleotide sequence at least about 90% identical to CGGCCGC. In some embodiments, the upper stem comprises a nucleotide sequence at least about 95% identical to CGGCCGC. In some embodiments, the upper stem comprises a nucleotide sequence at least about 99% identical to CGGCCGC. In some embodiments, the upper stem comprises a nucleotide sequence is identical to CGGCCGC.

In some embodiments, the upper stem comprises a nucleotide sequence at least about 80% identical to CGCGC. In some embodiments, the upper stem comprises a nucleotide sequence at least about 85% identical to CGCGC. In some embodiments, the upper stem comprises a nucleotide sequence at least about 90% identical to CGCGC. In some embodiments, the upper stem comprises a nucleotide sequence at least about 95% identical to CGCGC. In some embodiments, the upper stem comprises a nucleotide sequence at least about 99% identical to CGCGC. In some embodiments, the upper stem comprises a nucleotide sequence is identical to CGCGC.

In some embodiments, the upper stem comprises a nucleotide sequence at least about 80% identical to CGAU. In some embodiments, the upper stem comprises a nucleotide sequence at least about 85% identical to CGAU. In some embodiments, the upper stem comprises a nucleotide sequence at least about 90% identical to CGAU. In some embodiments, the upper stem comprises a nucleotide sequence at least about 95% identical to CGAU. In some embodiments, the upper stem comprises a nucleotide sequence at least about 99% identical to CGAU. In some embodiments, the upper stem comprises a nucleotide sequence is identical to CGAU.

In some embodiments, the stem loop comprises GC base pairs in the lower stem.

In some embodiments, the lower stem does not comprise GC base pairs.

In some embodiments, the upper stem does not comprise a GC base pair.

In some embodiments, the upper the stem comprises at least 1, 2, 3, 4, 5, or 6, 7, 8, 9, 10, 11, or 12 GC base pairs. In some embodiments, the stem comprises at least 1 GC base pair. In some embodiments, the stem comprises at least 2 GC base pairs. In some embodiments, the stem comprises at least 3 GC base pairs. In some embodiments, the stem comprises at least 4 GC base pairs. In some embodiments, the stem comprises at least 2 GC base pairs. In some embodiments, the stem comprises at least 5 GC base pairs. In some embodiments, the stem comprises at least 6 GC base pairs. In some embodiments, the stem comprises at least 7 GC base pairs. In some embodiments, the stem comprises at least 8 GC base pairs. In some embodiments, the stem comprises at least 9 GC base pairs. In some embodiments, the stem comprises at least 10 GC base pairs. In some embodiments, the stem comprises at least 11 GC base pairs. In some embodiments, the stem comprises at least 12 GC base pairs.

In some embodiments, ligating the first and the second RNA results in a yield of at least 60%, 70%, 80%, 90%, or more than 95% of full length product. In some embodiments, the first and the second RNA results in a yield of at least 60% of full length product. In some embodiments, the first and the second RNA results in a yield of at least 70% of full length product. In some embodiments, the first and the second RNA results in a yield of at least 80% of full length product. In some embodiments, the first and the second RNA results in a yield of at least 90% of full length product. In some embodiments, the first and the second RNA results in a yield of at least 95% of full length product. In some embodiments, the first and the second RNA results in a yield more than 95% of full length product.

In some embodiments, the gRNA is produced at a quantity of at least 1 gram.

In some embodiments, gRNA is produced at a quantity of at least 5 grams, 10 grams, 20 grams, 30 grams, 40 grams, 50 grams, 60 grams, 70 grams, 80 grams, 90 grams, or 100 grams. Accordingly, in some embodiments, gRNA is produced at a quantity of at least 5 grams. In some embodiments, gRNA is produced at a quantity of at least 10 grams. In some embodiments, gRNA is produced at a quantity of at least 20 grams. In some embodiments, gRNA is produced at a quantity of at least 30 grams. In some embodiments, gRNA is produced at a quantity of at least 40 grams. In some embodiments, gRNA is produced at a quantity of at least 50 grams. In some embodiments, gRNA is produced at a quantity of at least 60 grams. In some embodiments, gRNA is produced at a quantity of at least 70 grams. In some embodiments, gRNA is produced at a quantity of at least 80 grams. In some embodiments, gRNA is produced at a quantity of at least 90 grams. In some embodiments, gRNA is produced at a quantity of at least 100 grams.

In some embodiments, the gRNA is produced at a quantity of less than 1 gram.

In some embodiments, the gRNA is produced at a quantity of about 0.05 grams, 0.1 grams, 0.2 grams, 0.3 grams, 0.4 grams, 0.5 grams, 0.6 grams, 0.7 grams, 0.8 grams, or 0.9 g. In some embodiments, the gRNA is produced at a quantity of about 0.05 grams. In some embodiments, the gRNA is produced at a quantity of about 0.1 grams. In some embodiments, the gRNA is produced at a quantity of about 0.2 grams. In some embodiments, the gRNA is produced at a quantity of about 0.3 grams. In some embodiments, the gRNA is produced at a quantity of about 0.4 grams. In some embodiments, the gRNA is produced at a quantity of about 0.5 grams. In some embodiments, the gRNA is produced at a quantity of about 0.6 grams. In some embodiments, the gRNA is produced at a quantity of about 0.7 grams. In some embodiments, the gRNA is produced at a quantity of about 0.8 grams. In some embodiments, the gRNA is produced at a quantity of about 0.9 grams.

In some embodiments, the method produces gRNA at a purity of about 50%, 60%, 70%, 80%, 90%, or more than 90%. In some embodiments, the method produces gRNA at a purity of about 50%. In some embodiments, the method produces gRNA at a purity of about 60%. In some embodiments, the method produces gRNA at a purity of about 70%. In some embodiments, the method produces gRNA at a purity of about 80%. In some embodiments, the method produces gRNA at a purity of about 90%. In some embodiments, the method produces gRNA at a purity of more than 90%.

In some embodiments, the first RNA is synthesized in a 3′ to 5′ direction.

In some embodiments, the second RNA is synthesized in a 3′ to 5′ direction.

In some embodiments, the gRNA has a length of about 100 nucleotides, about 125 nucleotides, about 150 nucleotides, about 175 nucleotides, about 200 nucleotides, or greater than about 200 nucleotides. In some embodiments, the gRNA has a length of about 100 nucleotides. In some embodiments, the gRNA has a length of about 125 nucleotides. In some embodiments, the gRNA has a length of about 150 nucleotides. In some embodiments, the gRNA has a length of about 175 nucleotides. In some embodiments, the gRNA has a length of about 200 nucleotides. In some embodiments, the gRNA has a length of greater than 200 nucleotides.

In some embodiments, the loop comprises 4, 5, 6, 7, 8, 9, 10, 11, 12, 13, 14, 15 or 16 nucleotides. In some embodiments, the loop comprises 4 nucleotides, also referred to herein as a tetraloop. In some embodiments, the loop comprises 5 nucleotides. In some embodiments, the loop comprises 6 nucleotides. In some embodiments, the loop comprises 7 nucleotides. In some embodiments, the loop comprises 8 nucleotides. In some embodiments, the loop comprises 9 nucleotides. In some embodiments, the loop comprises 10 nucleotides. In some embodiments, the loop comprises 11 nucleotides. In some embodiments, the loop comprises 12 nucleotides. In some embodiments, the loop comprises 13 nucleotides. In some embodiments, the loop comprises 14 nucleotides. In some embodiments, the loop comprises 15 nucleotides. In some embodiments, the loop comprises 16 nucleotides.

In some embodiments, ligating the first RNA and the second RNA occurs at a ligation site that is at least about 3 base pairs from the loop.

In some embodiments, the ligation site is 1, 2, 3, 4, 5, 6, or 10 base pairs from the loop. In some embodiments, the ligation site is 1 base pair from the loop. In some embodiments, the ligation site is 2 base pairs from the ligation loop. In some embodiments, the ligation site is 3 base pairs from the ligation loop. In some embodiments, the ligation site is 4 base pairs from the ligation loop. In some embodiments, the ligation site is 5 base pairs from the ligation loop. In some embodiments, the ligation site is 6 base pairs from the ligation loop. In some embodiments, the ligation site is 7 base pairs from the ligation loop. In some embodiments, the ligation site is 8 base pairs from the ligation loop. In some embodiments, the ligation site is 9 base pairs from the ligation loop. In some embodiments, the ligation site is 10 base pairs from the ligation loop.

In some embodiments, the first and/or second RNA comprises one or more backbone modifications.

In some embodiments, the one or more backbone modifications comprises a 2′ O-methyl or a phosphorothioate modification. Accordingly, in some embodiments, the one or more backbone modifications comprises a 2′ O-methyl modification. In some embodiments, the one or more backbone modifications comprises a phosphorothioate modification.

In some embodiments, the one or more backbone modifications is selected from 2′-O-methyl 3′-phosphorothioate, 2′O-methyl, 2′-ribo 3′-phosphorothioate, deoxy, or 5′ phosphate modification. Accordingly, in some embodiments, the one or more backbone modifications comprises a 2′-O-methyl 3′-phosphorothioate modification. In some embodiments, the one or more modifications comprises a 2′-ribo 3′-phosphorothioate modification. In some embodiments, the one or more modifications comprises a deoxy modification. In some embodiments, the one or more modifications comprises a 5′ phosphate modification.

In some embodiments, the one or more modifications are present at the site of ligation.

In some embodiments, the one or more modifications are present in the donor RNA and/or the acceptor RNA. Accordingly, in some embodiments, the one or more modifications are present in the donor RNA. In some embodiments, the one or more modifications are present in the acceptor RNA. In some embodiments, the one or more modifications are present in both the donor and the acceptor RNA.

In some embodiments, the 3′ and/or the 5′ end of the donor RNA has one or more backbone modifications. Accordingly, in some embodiments, the 3′ end of the donor RNA has one or more backbone modifications. In some embodiments, the 5′ end of the donor RNA has one or more backbone modifications

In some embodiments, the 3′ and/or the 5′ end of the acceptor RNA has one or more backbone modifications. Accordingly, in some embodiments, the 3′ end of the acceptor RNA has one or more backbone modifications. In some embodiments, the 5′ end of the acceptor RNA has one or more backbone modifications.

In some embodiments, the concentration of the first and/or second RNA is between about 1 g/L and 5 g/L.

In some embodiments, the concentration of the first and/or second RNA is about 1 g/L. In some embodiments, the concentration of the first and/or second RNA is about 2 g/L. In some embodiments, the concentration of the first and/or second RNA is about 3 g/L. In some embodiments, the concentration of the first and/or second RNA is about 4 g/L. In some embodiments, the concentration of the first and/or second RNA is about 5 g/L.

In some embodiments, a composition produced by a method described herein is provided comprising a first RNA comprising a phosphate at a 5′ terminus and a second RNA comprising a variable protospacer region, wherein the first and the second RNA are non-covalently bound.

In some embodiments, a composition produced by a method described herein is provided comprising a first RNA comprising a phosphate at a 5′ terminus and a second RNA comprising a variable protospacer region, and wherein the first and the second RNA are bound to a ligase.

In some embodiments, the ligase is a T4 RNA ligase 2.

In some aspects, a composition is provided comprising an RNA comprising a nucleotide sequence at least about 80% identical to CGAUACGACAGAAC. In some embodiments, a composition is provided comprising an RNA comprising a nucleotide sequence at least about 85% identical to CGAUACGACAGAAC. In some embodiments, a composition is provided comprising an RNA comprising a nucleotide sequence at least about 90% identical to CGAUACGACAGAAC. In some embodiments, a composition is provided comprising an RNA comprising a nucleotide sequence at least about 95% identical to CGAUACGACAGAAC. In some embodiments, a composition is provided comprising an RNA comprising a nucleotide sequence identical to CGAUACGACAGAAC.

In some aspects, a composition is provided comprising an RNA comprising a nucleotide sequence at least about 80% identical to CGCCG. In some embodiments, a composition is provided comprising an RNA comprising a nucleotide sequence at least about 85% identical to CGCCG. In some embodiments, a composition is provided comprising an RNA comprising a nucleotide sequence at least about 90% identical to CGCCG. In some embodiments, a composition is provided comprising an RNA comprising a nucleotide sequence at least about 95% identical to CGCCG. In some embodiments, the nucleotide sequence is identical to CGCCG.

In some aspects, a composition is provided comprising an RNA comprising a nucleotide sequence at least about 80% identical to CGGCCGC. In some embodiments, a composition is provided comprising an RNA comprising a nucleotide sequence at least about 85% identical to CGGCCGC. In some embodiments, a composition is provided comprising an RNA comprising a nucleotide sequence at least about 90% identical to CGGCCGC. In some embodiments, a composition is provided comprising an RNA comprising a nucleotide sequence at least about 95% identical to CGGCCGC. In some embodiments, the nucleotide sequence is identical to CGGCCGC.

In some aspects, a composition is provided comprising an RNA comprising a nucleotide sequence at least about 80% identical to CGCGC. In some embodiments, a composition is provided comprising an RNA comprising a nucleotide sequence at least about 85% identical to CGCGC. In some embodiments, a composition is provided comprising an RNA comprising a nucleotide sequence at least about 90% identical to CGCGC. In some embodiments, a composition is provided comprising an RNA comprising a nucleotide sequence at least about 95% identical to CGCGC. In some embodiments, the nucleotide sequence is identical to CGCGC.

In some embodiments, a kit is provided comprising a composition described herein.

In some aspects, a kit is provided comprising a first RNA comprising trans-activating RNA (tracrRNA) sequence, a second RNA comprising a variable protospacer region, and a ligase.

In some embodiments, the kit comprises a T4 RNA Ligase 2.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about. As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of AT to GC in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

Base Editing Activity: By “base editing activity” is meant acting to chemically alter a base within a polynucleotide (e.g., by deaminating the base). In one embodiment, a first base is converted to a second base. In one embodiment, the base editing activity is cytidine deaminase activity, e.g., converting target CG to TA. In another embodiment, the base editing activity is adenosine or adenine deaminase activity, e.g., converting AT to GC. In another embodiment, the base editing activity is cytidine deaminase activity, e.g., converting target CG to TA and adenosine or adenine deaminase activity, e.g., converting AT to GC.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: By “complementary” or “complementarity” is meant that a nucleic acid can form hydrogen bond(s) with another nucleic acid sequence by either traditional Watson-Crick or Hoogsteen base pairing. Complementary base pairing includes not only G-C and A-T base pairing, but also includes base pairing involving universal bases, such as inosine. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). To determine that a percent complementarity is of at least a certain percentage, the percentage of contiguous residues in a nucleic acid molecule that can form hydrogen bonds (e.g., Watson-Crick base pairing) with a second nucleic acid sequence is calculated and rounded to the nearest whole number (e.g., 12, 13, 14, 15, 16, or 17 nucleotides out of a total of 23 nucleotides in the first oligonucleotide being based paired to a second nucleic acid sequence having 23 nucleotides represents 52%, 57%, 61%, 65%, 70%, and 74%, respectively; and has at least 50%, 50%, 60%, 60%, 70%, and 70% complementarity, respectively). As used herein, “substantially complementary” refers to complementarity between the strands such that they are capable of hybridizing under biological conditions. Substantially complementary sequences have 60%, 70%, 80%, 90%, 95%, or even 100% complementarity. Additionally, techniques to determine if two strands are capable of hybridizing under biological conditions by examining their nucleotide sequences are well known in the art.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity and/or that binds to a target site on a nucleic acid specified by a RNA guide. In different embodiments, a CRISPR effector has endonuclease activity, nickase activity, exonuclease activity, transposase activity, and/or excision activity. In other embodiments, the CRISPR effector is nuclease inactive.

crRNA: The term “CRISPR RNA” or “crRNA,” as used herein, refers to a RNA molecule including a guide sequence used by a CRISPR effector to target a specific nucleic acid sequence. Typically, crRNAs contain a sequence that mediates target recognition and a sequence that forms a duplex with a tracrRNA. In some embodiments, the crRNA: tracrRNA duplex binds to a CRISPR effector.

Duplex: As used herein, “duplex” refers to a double helical structure formed by the interaction of two single stranded nucleic acids. A duplex is typically formed by the pairwise hydrogen bonding of bases, i.e., “base pairing”, between two single stranded nucleic acids which are oriented antiparallel with respect to each other. Base pairing in duplexes generally occurs by Watson-Crick base pairing, e.g., guanine (G) forms a base pair with cytosine (C) in DNA and RNA, adenine (A) forms a base pair with thymine (T) in DNA, and adenine (A) forms a base pair with uracil (U) in RNA. Conditions under which base pairs can form include physiological or biologically relevant conditions (e.g., intracellular: pH 7.2, 140 mM potassium ion; extracellular pH 7.4, 145 mM sodium ion). Furthermore, duplexes are stabilized by stacking interactions between adjacent nucleotides. As used herein, a duplex may be established or maintained by base pairing or by stacking interactions. A duplex is formed by two complementary nucleic acid strands, which may be substantially complementary or fully complementary. Single-stranded nucleic acids that base pair over a number of bases are said to “hybridize.”

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Hybridize: By “hybridize” is meant to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). Hybridization occurs by hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Indel: As used herein, the term “indel” refers to insertion or deletion of bases in a nucleic acid sequence. It commonly results in mutations and is a common form of genetic variation.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. A PAM may be required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Prime editing guide RNA: The term “prime editing guide RNA” or “pegRNA” refers to a type of guide RNA that both specifies a target site and encodes a desired edit. Prime editing guide RNAs (pegRNAs) are known in the art and have been described previously, for example in Anzalone A. V., “Search-and-replace genome editing without double-strand breaks or donor DNA” Nature. 2019 Oct. 21. doi: 10.1038/s41586-019-1711-4, the entire contents of which are incorporated herein by reference.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term RNA guide refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Splint: The term “splint” refers to refers to a single stranded RNA or DNA or other polymer that is capable of hybridizing with at least two, three or more single stranded RNA nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA,” “single guide RNA,” or “guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWING

Drawings are for illustration purposes only; not for limitation.

FIG. 1 is a schematic which shows the standard chemical synthesis of synthetic RNA. Synthetic RNA is typically synthesized through sequence-controlled polymerization on a solid support. Chemical synthesis is performed in cycles, each comprising various steps as illustrated in the schematic of FIG. 1.

FIG. 2 is a general schematic that shows sgRNA interacting with a target DNA sequence. The schematic illustrates various motifs present in the sgRNA, including the spacer region, the stem loop comprised of the lower stem, tetraloop, and bulge region, the nexus motif, and a series of hairpin motifs.

FIG. 3, panel A shows two general approaches for the synthesis of sgRNA using the ligation-based method. In one approach (1) the ligation occurs at the loop portion of the stem loop. In the second approach (2) the ligation occurs at the helix of the stem loop. In each approach, the stem loop is extended and is used to associate the sections for enzymatic ligation. FIG. 3, panel B depicts a schematic HPLC graph that shows separation between the RNA fragments and the gRNA produced by ligating the fragments, represented by peaks. After ligation a final purification step is performed using HPLC to remove RNA fragments that did not ligate. Complete separation of the RNA fragments from the full-length product (FLP) is possible.

FIG. 4 is a schematic which shows an example of a typical click chemistry reaction used in drug synthesis. Prior methods used chemical ligation using “click chemistry” to combine RNA fragments into full length sgRNA.

FIG. 5, panels A-C, depict various substrates that are used for enzymatic ligation. FIG. 5, panel A, depicts two RNA oligos that are associated on a splint. In this scenario a nick (e.g., a joining point between a first RNA and a second RNA) will be sealed by a ligase, resulting in a natural phosphodiester backbone linkage. FIG. 5, panel B depicts two RNA oligos that have partial complementarity with each other, and base pair together forming a stem loop structure. Enzymatic ligations can be performed with high efficiency in the loop section of a stem loop. FIG. 5, panel C depicts two RNAs that base pair with a splint. Ligations can be performed in various RNA associations, and can be performed with no pre-association required.

FIG. 6, panel A depicts the sequence and the configuration associated with the most commonly used sgRNA. FIG. 6, panel B shows two representative RNA sequences and an associated ligation site for loop ligation. FIG. 6, panel C shows two representative RNA sequences and ligation site for helix ligation. Sequences are as follows:

Panel A:
XXXXXXXXXXXXXXXXXXXXGUUUUAGAGCUAGAAAUAGCAAGUUAAAAUA
AGGCUAGUCCGUUAUCAACUUGAAAAAGUGGGACCGAGUCGGUGCAGACUU
CUCCACAGGAGUCAGGUGCAC
Panel B:
XXXXXXXXXXXXXXXXXXXXGUUUUAGAGCUAUGCUGUCUUGCUCGA
pUACAAGACAGCAUAGCAAGUUAAAAUAAGGCUAGUCCGUUAUCAACU
UGAAAAAGUGGCACCGAGUCGGUGCUUUU
Panel C:
XXXXXXXXXXXXXXXXXXXXGUUUUAGAGCUAUGCUGU
pCUUGGAAACAAGACAGCAUAGCAAGUUAAAAUAAGGCUAGUCCGUUA
UCAACUUGAAAAAGUGGCACCGAGUCGGUGCUUUU

Where X is any nucleotide and “p” indicates a free phosphate where the RNAs are not covalently linked as illustrated in the figure.

FIG. 7, panel A depicts sequences of fragments used in ligation experiment. Both the acceptor and the donor sequences are shown. Base code: A, adenosine; G, guanosine; U, uridine; C, cytidine; mA, 2′-O-methyl-adenosine; mU, 2′-O-methyl-uridine; mC, 2′-O-methyl-cytidine; pC, 5′-phosphorylated cytidine. Panel B shows a proposed structure of the pre-ligated complex. Acceptor and Donor sequences are shown and correspond to the Acceptor and Donor sequences shown in FIG. 7 panel A. Phosphates are represented as circles. Panel C shows chromatograms showing: 1, acceptor fragment; 2, donor fragment; 3, products of reaction between acceptor and donor fragment with T4 RNA Ligase 2. Reaction contained 10 μM donor fragment, 10 μM acceptor fragment, 40 μL of 1× T4 RNA Ligase 2 Reaction Buffer (NEB), and 20 units of T4 RNA Ligase 2, and was performed at 37° C.

FIG. 8, panel A, depicts RNA fragment designs for RNA donor 1 (Dnr-01), RNA acceptor 1 (Acp-01) designs and the ligation complex. Dnr-01 and Acp-01 sequences are shown in Table 4. The letter “P” indicates position of phosphate and ligation. FIG. 8, panel B, depicts HPLC chromatograms for reactions between Acp-1 and Dnr-1 with and without T4 RNA ligase 2. “FLP” stands for “full-length product.”

FIG. 9, panel A, depicts RNA fragment designs for RNA acceptor 2 (Acp-02), RNA donor 2 (Dnr-02), and ligation complex. The letter “P” indicates position of phosphate and ligation. FIG. 9, panel B, depicts an HPLC chromatograms for reactions between Acp-02 and Dnr-02 with and without the ligase T4 RNA ligase 1. Acp-02 and Dnr-02 sequences are shown in Table 4. “FLP” stands for “full-length product.”

FIG. 10, panel A, depicts RNA fragment designs for RNA acceptor 3 (Acp-03), RNA acceptor 3 (Dnr-03), and ligation complex. The letter “P” indicates position of phosphate and ligation. FIG. 10, panel B, depicts HPLC chromatograms for reactions between Acp-03 and Dnr-03 with and without T4 RNA Ligase 2. FIG. 10, panel C, depicts fragment designs for RNA acceptor 4 (Acp-04), RNA donor 4 (Dnr-04) and ligation complex. The letter “P” indicates position of ligation site. FIG. 10, panel D, depicts HPLC chromatograms for reactions between RNA acceptor 4 (Acp-04) and RNA donor 4 (Dnr-04) with and without T4 RNA Ligase 2. Acp-03 and Dnr-03 sequences are shown in Table 4. “FLP” stands for “full-length product.”

FIG. 11, panel A, depicts RNA fragment designs for RNA acceptor 5 (Acp-05), RNA donor 5 (Dnr-05), and ligation complex. The letter “P” indicates position of ligation site. FIG. 11, panel B, depicts HPLC chromatograms for reactions between Acp-05 and Dnr-05 with and without Ligase 2. FIG. 11, panel C, depicts RNA fragment designs for RNA acceptor 6 (Acp-06), RNA donor 6 (Dnr-06) and ligation complex. The letter “P” indicates position of ligation site. FIG. 11, panel D, depicts HPLC chromatograms for reactions between Acp-06 and Dnr-06 with and without T4 RNA Ligase 2. Acp-05 and Dnr-05 sequences are shown in Table 4. The letter “P” indicates position of ligation site. “FLP” stands for “full-length product.”

FIG. 12, panel A, depicts fragment designs for RNA acceptor 7 (Acp-07), RNA donor 7 (Dnr-07), and ligation complex. The letter “P” indicates position of ligation site. FIG. 12, panel B, depicts HPLC chromatograms for reactions between Acp-07 and Dnr-07 with and without T4 RNA Ligase 2. Acp-07 and Dnr-07 sequences are shown in Table 4. “FLP” stands for “full-length product.” Side product is produced in reaction is labeled with an “*.”

FIG. 13 is a graph that shows yield of reaction (“FLP”) as a function of starting fragment concentration (g/L). For these studies, RNA acceptor 5/RNA donor 5 (Acp/Dnr-05) and RNA acceptor 6/RNA donor 6 (Acp/Dnr-6) were used. “FLP” stands for “full-length product.”

FIG. 14, panel A, depicts fragment designs for extensively modified fragments: RNA acceptor 8 (Acp-08), RNA donor 8 (Dnr-08), and ligation complex. Acp-08 and Dnr-08 sequences are shown in Table 4. The highlighted/shaded nucleotides in panel A indicate positions modified with 2 O-methyl modifications. The letter “P” indicates position of ligation site. FIG. 14, panel B, depicts HPLC chromatograms of these reactions. The letter “P” indicates position of phosphate and ligation. FIG. 14, panel B, shows chromatograms from the reactions in the presence (solid line) and absence (dotted line) of ligase (T4 RNA Ligase 2). “FLP” stands for full length product.

FIG. 15 is a graph that shows percent editing in fibroblast cells using an adenine base editor (ABE) and one of three guide RNAs (AD-08, AD-05, AD-06) which were synthesized using a self-templating ligation method.

FIG. 16 is a schematic that shows a sequence and secondary structure of Bacillus hisashii, bhCas12b sgRNA. The schematic shows regions labeled as “A,” “B,” and “C” which indicate hairpin loop structures that can be targeted as positions to split the sgRNA. The letter “N” in the sequence indicates any nucleobase.

### DETAILED DESCRIPTION

The present invention provides methods of producing synthetic RNAs. Any synthetic RNA can be created with the methods described herein. For example, in some embodiments, the ligation methods provided can be used for the production of guide RNAs (gRNAs) that are useful in modifying a specific locus in a target DNA or RNA when used with a site-directed modifying polypeptide such as Cas9, Cpf1, SaCas, Cas12, Cas13, base editor and prime editor among others. The inventors have surprisingly discovered a method of producing gRNAs from RNA fragments that results in the production of gRNAs that have high purity, integrity and final (post-purified) yield.

Various aspects of the invention are described in detail in the following sections. The use of sections is not meant to limit the invention. Each section can apply to any aspect of the invention. In this application, the use of “or” means “and/or” unless stated otherwise.

Guide RNA (gRNA)

A gRNA comprises a polynucleotide sequence complementary to a target sequence. The gRNA hybridizes with the target nucleic acid sequence and directs sequence-specific binding of a CRISPR complex to the target nucleic acid. In some embodiments, an RNA guide has 50%, 60%, 70%, 75%, 80%, 85%, 90%, 95%, 96%, 97%, 98%, 99% or 100% complementarity to a target nucleic acid sequence.

In some embodiments, the gRNA of the present invention is between about 50 nucleotides and 250 nucleotides. Accordingly, in some embodiments, the gRNA of the present invention is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides long. In some embodiments, the gRNA of is between about 50 and 75 nucleotides long. In some embodiments, the gRNA is between about 75 and 100 nucleotides long. In some embodiments, the gRNA is between about 100 and 125 nucleotides long. In some embodiments, the gRNA is between about 125 and 150 nucleotides long. In some embodiments, the gRNA is between about 150 and 175 nucleotides long. In some embodiments, the gRNA is between about 175 and 200 nucleotides long. In some embodiments, the gRNA is between about 200 and 225 nucleotides long. In some embodiments, the gRNA is between about 225 and 250 nucleotides long. In some embodiments, the gRNA is a “prime editing guide RNA” or a “pegRNA.” See Anzalone et al., Nature, 2019 Oct. 21, the contents of which are incorporated herein by reference.

In some embodiments, the gRNA comprises a ligated crRNA and a tracrRNA. Various crRNA and tracrRNA sequences are known in the art, for example those associated with several type II CRISPR-Cas9 systems (e.g., WO2013/176772), Cpf1, SaCas, Cas12, and prime editing Cas among others.

A gRNA can be designed to target any target sequence. Optimal alignment is determined using any algorithm for aligning sequences, including the Needleman-Wunsch algorithm, Smith-Waterman algorithm, Burrows-Wheeler algorithm, ClustlW, ClustlX, BLAST, Novoalign, SOAP, Maq, and ELAND.

In some embodiments, a gRNA is designed to target to a unique target sequence within the genome of a cell. In some embodiments, a gRNA is designed to lack a PAM sequence. In some embodiments, a gRNA sequence is designed to have optimal secondary structure using a folding algorithm including mFold or Geneious. In some embodiments, expression of gRNAs may be under an inducible promoter, e.g. hormone inducible, tetracycline or doxycycline inducible, arabinose inducible, or light inducible.

In some embodiments, the gRNA sequence is a “dead crRNAs,” “dead guides,” or “dead guide sequences” that can form a complex with a CRISPR-associated protein and bind specific targets without any substantial nuclease activity.

In some embodiments, the gRNA is chemically modified in the sugar phosphate backbone or base. In some embodiments, the gRNA has one or more of the following modifications 2′O-methyl, 2′-F or locked nucleic acids to improve nuclease resistance or base pairing. In some embodiments, the gRNA may contain modified bases such as 2-thiouridiene or N6-methyladenosine.

In some embodiments, the gRNA is conjugated with other oligonucleotides, peptides, proteins, tags, dyes, or polyethylene glycol.

In some embodiments, the gRNA includes an aptamer or riboswitch sequence that binds specific target molecules due to their three-dimensional structure.

In some embodiments, the loop forming sequences are 3, 4, 5 or more nucleotides in length. In some embodiments, the loop has the sequence GAAA, AAAG, CAAA and/or AAAC.

In some embodiments, gRNA has two, three, four or five hairpins.

In some embodiments, gRNA includes a transcription termination sequence, which includes a polyT sequences comprising six nucleotides.

### Production of Synthetic Guide RNA

Described herein is a method of making synthetic RNA, such as guide RNA (gRNA). The described methods produces synthetic RNA, such as gRNA, that has high integrity and yield.

The ligation strategies described herein differ from previously-reported chemical ligation strategies used to synthesize synthetic RNAs, such as gRNAs, as the described strategies form natural phosphate linkage at the site of ligation. The advantage of using a segmented synthetic approach (as described herein) is that short sections of RNA can be produced with greater purity post-purification compared to full length gRNA. In this approach the 5′ acceptor is the smallest RNA fragment (about 30-50 nts) and can thus be purified to a high level before ligation. The 3′ donor is terminated with a phosphate that is required for synthesis and thus only the full-length fragment will be incorporated into the full length product (i.e., truncations are not substrates).

The advantages of the methods described herein is increased when considering gRNAs that are greater than 100 nts, such as pegRNA or Cas12b guides. The types of enzymatic ligations described herein are very high yielding (>80%) and the oligonucleotide starting material can be separated from ligated product with high selectivity, ensuring that full-length product is very pure. These types of enzymatic ligations are relatively inexpensive and scale well.

Self-Templating Approach to Producing Synthetic gRNA

In some aspects, the method of making synthetic gRNA comprises: providing a first and a second RNA having complementarity, wherein the complementarity allows for base pairing and the creation of a stem loop between the first and the second RNA; ligating the first and second RNA with a ligating enzyme within the stem loop, thus producing a synthetic gRNA. This allows for the use of a helix, or other structure, that is formed between the first RNA and the second RNA to template an enzymatic ligation of the two RNAs. In some embodiments, the length and sequence composition of the structure formed between the first and the second RNA is modified to promote non-covalent assembly and to create optimal ligation sites for enzymes compatible with RNA ligation.

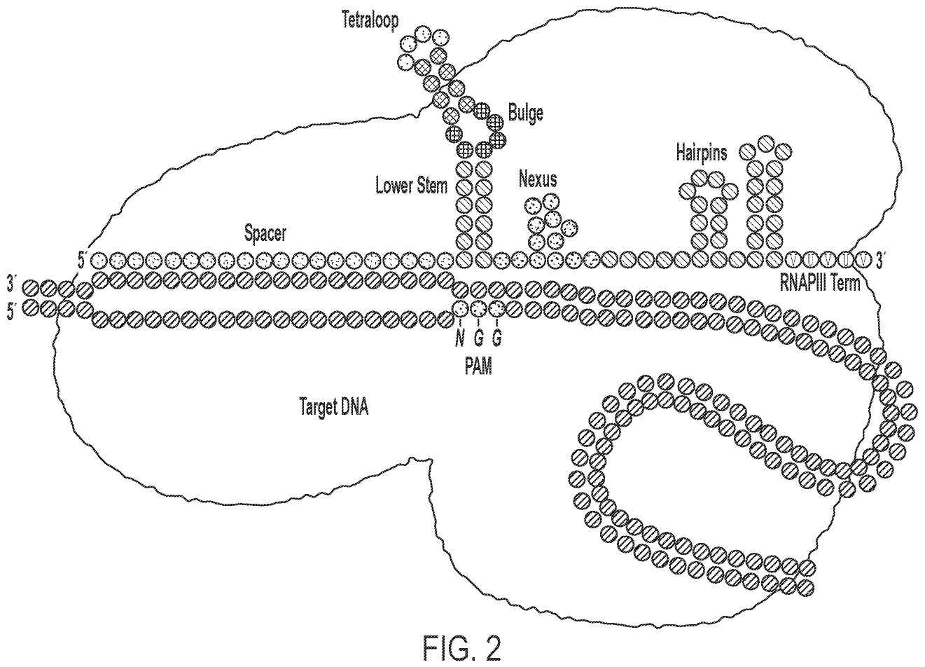

The complementarity can either be partial or perfect among a stretch of nucleotides of the first and the second RNA. The complementarity allows for base pairing between the complementary nucleotides. In regions where there is partial complementarity, the mismatched nucleotides would result in the formation of a bulge or a loop structure between the first and the second RNA molecule. Various structures can be formed between the first and the second RNA molecule based on hybridization between the two RNA molecules. Exemplary structures that can be formed between the first and the second RNA molecules are illustrated in FIG. 2. The ligation between the two RNA molecules can occur at a stem, helix, loop, overhang, blunt end or at a bulge.

Using this approach, a first RNA is synthesized with a phosphate at the 5′-terminus (termed donor) which is ligated to the 3′-terminus of a second RNA which comprises the variable protospacer region (termed acceptor) by one of multiple ligases.

In some embodiments, two or more RNA fragments are ligated using this approach. For example, in some embodiments, 2, 3, 4, 5, 6, 7, 8, 9, 10 or more RNA fragments are ligated using the self-templating approach. Accordingly, in some embodiments, the self-templating approach of producing a synthetic RNA comprises providing two or more RNA fragments; providing an oligonucleotide that has partial complementarity to the two or more RNA fragments, wherein the complementarity of the oligonucleotide allows for base pairing with the two or more RNA fragments; and providing a ligase to catalyze ligation between the two or more RNA fragments, thus producing a synthetic guide RNA.

Various ligases can be used with the methods described herein. For example, one or more of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, SplintR Ligase E. coli DNA Ligase, 9° N DNA Ligase, CircLigase, CircLigase II, DNA Ligase I, DNA Ligase III, and DNA Ligase IV can be used. In some embodiments, T4 RNA ligase 1 is used to ligate the first RNA and the second RNA at the terminal loop. In some embodiments, T4 RNA ligase 2 is used for ligating the first RNA and the second RNA within the stem formed between the first RNA and the second RNA.

Various kinds of ligation are possible using this approach, such as ligation within a terminal loop of a hairpin formed between the first RNA and the second RNA. Various ligases are suitable for ligation at the terminal loop of a hairpin formed, such as T4 RNA ligase 1. Another kind of ligation that is possible with this approach is ligation within the duplex formed between the first RNA and the second RNA. Various ligases are suitable for ligating at the duplex formed between the two RNAs, such as T4 RNA ligase 2 and DNA ligases.

In some embodiments, the first RNA is a tran-activating RNA (tracrRNA), and the second RNA is a clustered regularly interspersed short palindromic repeats (CRISPR) RNA (crRNA).

In some embodiments, the first RNA is between about 10 to about 100 nucleotides long. Accordingly, in some embodiments, the first RNA is between about 10 and 25 nucleotides long. In some embodiments, the first RNA is between about 25 and 40 nucleotides long. In some embodiments, the first RNA is between about 40 and 45 nucleotides long. In some embodiments, the first RNA is between about 45 and 60 nucleotides long. In some embodiments, the first RNA is between about 60 and 75 nucleotides long. In some embodiments, the first RNA is between about 75 and 90 nucleotides long. In some embodiments, the first RNA is between about 90 and 100 nucleotides long.

In some embodiments, the second RNA is between about 10 to about 100 nucleotides long. Accordingly, in some embodiments, the second RNA is between about 10 and 25 nucleotides long. In some embodiments, the second RNA is between about 25 and 40 nucleotides long. In some embodiments, the second RNA is between about 40 and 45 nucleotides long. In some embodiments, the second RNA is between about 45 and 60 nucleotides long. In some embodiments, the second RNA is between about 60 and 75 nucleotides long. In some embodiments, the second RNA is between about 75 and 90 nucleotides long. In some embodiments, the second RNA is between about 90 and 100 nucleotides long.

Splint Templating Approach in the Production of Synthetic RNAs

In some embodiments, a splint is used in the production of the synthetic RNAs. The use of a splint allows for one or more RNA molecules to be brought into physical proximity for the reaction using a splint as a template. When more than two RNAs are to be joined, the use of splints facilitates the production of the synthetic RNAs.

The splints can be any suitable polymer that is capable of bringing the one or more RNA molecules in close proximity can be used. For example, in some embodiments, the splint is an RNA molecule or a DNA molecule.

In some embodiments, the splint has complementarity to sections of the first RNA and the second RNA. The complementarity can either be partial or perfect. Accordingly, in some embodiments, a method of producing a synthetic RNA, such as a guide RNA, is provided comprising providing a first RNA comprising a 5′ phophate; providing a second RNA comprising a free 3′-hydoxyl; providing an oligonucleotide that has partial complementarity to the first RNA and the second RNA, wherein the complementarity of the oligonucleotide allows for base pairing with the first and the second RNA; and providing a ligase to catalyze ligation between the first and the second RNA, thus producing a gRNA.

In some embodiments, the splint has no complementarity to the sections of the first RNA and the second RNA that will be coupled. Accordingly, in some embodiments, a method of producing a synthetic RNA, such as a guide RNA, is provided comprising providing a first RNA comprising a 5′ phosphate; providing a second RNA comprising a free 3′-hydoxyl; providing an oligonucleotide that has no complementarity to nucleotides of the first RNA and the second RNA that will be coupled; and providing a ligase to catalyze ligation between the first and the second RNA, thus producing a gRNA.

Non-Templated Approach to Produce Synthetic RNAs

In some embodiments a non-templated approach is used to produce synthetic RNAs, such as guide RNAs.

In some embodiments of the non-templated approach, a first RNA is provided that has a 5′ phosphate (such as a 5′ monophosphate), and a second RNA is provided that comprises a blocked 3′ end (such as a blocked 3′ OH). The purpose of blocking the 3′ OH of the second RNA is so that the second RNA cannot cyclize through an untemplated mechanism when ligation occurs. For example, using this non-templated approach can comprise a second RNA comprising 3′ hydroxyl of the 3′ terminal end of the donor molecule which is chemically blocked or removed (e.g., dideoxynucleotide) and an enzyme (particularly by T4 RNA Ligase 1) would catalyze proper ligation between the first RNA and the second RNA. In some embodiments, this ligation strategy is carried out at a high concentration.

Thus, in some aspects, the non-templating approach of producing a synthetic RNA comprises providing a first RNA comprising a 5′-monophosphate; providing a second RNA comprising a blocked 3′ end; and providing a ligase to catalyze ligation between the first and the second RNA, thus producing a gRNA.

Chemically Modified RNA

In some embodiments, the first RNA and/or the second RNA comprises a chemical modification to its backbone or to one or more of its bases. For example, chemically modified RNA can comprise chemical synthesis can be used to install highly modified monomers including modified sugars, bases, backbones or functional groups that do not resemble natural nucleotides.

Accordingly, in some embodiments, the first RNA and/or the second RNA comprises a modified base. In some embodiments, the modified RNA include one or more of the following 2′-O-methoxy-ethyl bases (2′-MOE) such as 2-MethoxyEthoxy A, 2-MethoxyEthoxy MeC, 2-MethoxyEthoxy G, 2-MethoxyEthoxy T. Other modified bases include for example, 2′-O-Methyl RNA bases, and fluoro bases. Various fluoro bases are known, and include for example, Fluoro C, Fluoro U, Fluoro A, Fluoro G bases. Various 2′OMethyl modifications can also be used with the methods described herein. For example, the following RNA comprising one or more of the following 2′OMethyl modifications can be used with the methods described: 2′-OMe-5-Methyl-rC, 2′-OMe-rT, 2′-OMe-rI, 2′-OMe-2-Amino-rA, Aminolinker-C6-rC, Aminolinker-C6-rU, 2′-OMe-5-Br-rU, 2′-OMe-5-I-rU, 2-OMe-7-Deaza-rG.

In some embodiments, the first RNA and/or second RNA comprises one or more of the following modifications: phosphorothioates, 2′O-methyl, 2′ fluoro (2′F), DNA.

In some embodiments, the first RNA and/or the second RNA comprises 2′OMe modifications at the 3′ and 5′-ends.

In some embodiments, the first RNA and/or second RNA comprises one or more of the following modifications: 2′-O-2-Methoxyethyl (MOE), locked nucleic acids, bridged nucleic acids, unlocked nucleic acids, peptide nucleic acids, morpholino nucleic acids.

In some embodiments, the first RNA and/or second RNA comprises one or more of the following base modifications: 2,6-diaminopurine, 2-aminopurine, pseudouracil, N1-methyl-psuedouracil, 5′ methyl cytosine, 2′pyrimidinone (zebularine), thymine.

Other modified bases include for example, 2-Aminopurine, 5-Bromo dU, deoxyUridine, 2,6-Diaminopurine (2-Amino-dA), Dideoxy-C, deoxylnosine, Hydroxymethyl dC, Inverted dT, Iso-dG, Iso-dC, Inverted Dideoxy-T, 5-Methyl dC, 5-Methyl dC, 5-Nitroindole, Super T®, 2′-F-r(C,U), 2′-NH2-r(C,U), 2,2′-Anhydro-U, 3′-Desoxy-r(A,C,G,U), 3′-O-Methyl-r(A,C,G,U), rT, rI, 5-Methyl-rC, 2-Amino-rA, rSpacer (Abasic), 7-Deaza-rG, 7-Deaza-rA, 8-Oxo-rG, 5-Halogenated-rU, N-Alkylated-rN.

Other chemically modified RNA can be used herein. For example, the first RNA and/or second RNA can comprise a modified base such as, for example, 5′, Int, 3′ Azide (NHS Ester); 5′ Hexynyl; 5′, Int, 3′ 5-Octadiynyl dU; 5′, Int Biotin (Azide); 5′, Int 6-FAM (Azide); and 5′, Int 5-TAMRA (Azide). Other examples of RNA nucleotide modifications that can be used with the methods described herein include for example phosphorylation modifications, such as 5′-phosphorylation and 3′-phosphorylation. The RNA can also have one or more of the following modifications: an amino modification, biotinylation, thiol modification, alkyne modifier, adenylation, Azide (NHS Ester), Cholesterol-TEG, and Digoxigenin (NHS Ester).

Acceptor and Donor RNA Ligations

In some embodiments, the acceptor RNA and the donor RNA are ligated at a ligation site that is at a set distance from the loop formed between the acceptor RNA and the donor RNA. For example, the ligation site is at least 1, 2, 3, 4, 5, 6, 7, 8, 9, or 10 base pairs from the loop formed between the acceptor RNA and donor RNA. In some embodiments, the ligation site is 2 or 3 base pairs from the loop. The loop structures formed between the acceptor RNA and the donor RNA can vary in terms of length. For example, the loop formed between the acceptor RNA and the donor RNA can be 4, 5, 6, 7, 8, 9, 10, 11, 12, 13, 14, 15 or 16 nucleotides long. In some embodiments, the loop length is 4. A loop length of 4 is called a tetraloop herein. In some embodiments, the loop comprises 7 nucleotides.

In some embodiments, the acceptor and the donor RNA are ligated at a ligation site that is at a set distance from the bulge formed between the acceptor RNA and the donor RNA. For example, ligating the acceptor RNA and the donor RNA occurs at a ligation site that is at least about 3, 4, 5, 6, 7, 8, 10, 11 or 12 base pairs away from the bulge. In some embodiments, ligating the acceptor RNA and the donor RNA occurs at a ligation site that is 3, 4, 5, or 11 base pairs from the bulge.

The base pairing between the acceptor RNA and the donor RNA can occur at the lower stem and/or the upper stem. Thus, in some embodiments, the acceptor RNA and the donor RNA have nucleotide complementarity. The nucleotide complementarity can be partial, for example the complementarity between the acceptor RNA and the donor RNA can be from about 50% to about 99% complementarity. In some embodiments, the acceptor RNA and the donor RNA have nucleotides that have perfectly complementary.

In some embodiments, the acceptor RNA and the donor RNA are present at a ratio of about 0.5:1, 0.6:1, 0.7:1, 0.8:1, 0.9:1, 1:1, 1:0.9, 1:0.8, 1:0.7, 1:0.6, or 1:0.5.

In some embodiments, the methods described herein allows for the production of gRNA that has an improved yield as compared to gRNA produced using conventional synthetic methods. For example, in some embodiments, the gRNA produced in accordance with the methods described herein have about 50%, 55%, 60%, 65%, 70%, 75%, 80%, 85%, 90%, 95%, 99% or more improvement in yield as compared to conventional synthetic methods.

In some embodiments, the GC content of the upper and/or lower stem influences the yield, productivity and purity of the RNA ligation reaction.

In some embodiments, the acceptor RNA and the donor RNA does not comprise GC base pairs in the upper stem.

In some embodiments, a single donor fragment can be used with various acceptor fragments. In this manner, the donor fragment can serve as a universal donor fragment, which can be paired with one or more combinations of various acceptor fragments.

In some embodiments, the acceptor RNA and the donor RNA are engineered to contain GC base pairs in the upper stem. In some embodiments, the acceptor RNA and the donor RNA comprise at least 1, 2, 3, 4, 5, 6, 7, 8, 9, 10, 11, or 12 GC base pairs in the upper stem. In some embodiments, the acceptor RNA and the donor RNA comprise 2 GC nucleotides in the upper stem. Exemplary upper stem nucleotides described herein include:

(SEQ ID NO: 1)
CGAUACGACAGAAC;
(SEQ ID NO: 2)
CGCCG;
(SEQ ID NO: 3)
CGGCCGC;
(SEQ ID NO: 4)
CCGC ;
and
(SEQ ID NO: 5)
CGAU.

In some embodiments, the acceptor RNA and the donor RNA does not comprise GC base pairs in the lower stem. In some embodiments, acceptor RNA and donor RNA comprises GC base pairs in the lower stem.

In some embodiments, the concentration of the acceptor and the donor RNA is between about 1 g/L and 5 g/L. In some embodiments, the concentration of the acceptor and the donor RNA impacts the yield, productivity and purity of the resultant gRNA.

In some embodiments, the temperature at which the ligation reaction occurs influences the yield or productivity of the RNA ligation reaction. In some embodiments, the temperature at which the ligation reaction occurs is about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. or 40° C. Accordingly, in some embodiments, the temperature at which the ligation reaction occurs is about 15° C. In some embodiments, the temperature at which the ligation reaction occurs is about 16° C. In some embodiments, the temperature at which the ligation reaction occurs is about 17° C. In some embodiments, the temperature at which the ligation reaction occurs is about 18° C. In some embodiments, the temperature at which the ligation reaction occurs is about 19° C. In some embodiments, the temperature at which the ligation reaction occurs is about 20° C. In some embodiments, the temperature at which the ligation reaction occurs is about 21° C. In some embodiments, the temperature at which the ligation reaction occurs is about 22° C. In some embodiments, the temperature at which the ligation reaction occurs is about 23° C. In some embodiments, the temperature at which the ligation reaction occurs is about 24° C. In some embodiments, the temperature at which the ligation reaction occurs is about 25° C. In some embodiments, the temperature at which the ligation reaction occurs is about 26° C. In some embodiments, the temperature at which the ligation reaction occurs is about 27° C. In some embodiments, the temperature at which the ligation reaction occurs is about 28° C. In some embodiments, the temperature at which the ligation reaction occurs is about 29° C. In some embodiments, the temperature at which the ligation reaction occurs is about 30° C. In some embodiments, the temperature at which the ligation reaction occurs is about 31° C. In some embodiments, the temperature at which the ligation reaction occurs is about 32° C. In some embodiments, the temperature at which the ligation reaction occurs is about 33° C. In some embodiments, the temperature at which the ligation reaction occurs is about 34° C. In some embodiments, the temperature at which the ligation reaction occurs is about 35° C. In some embodiments, the temperature at which the ligation reaction occurs is about 36° C. In some embodiments, the temperature at which the ligation reaction occurs is about 37° C. In some embodiments, the temperature at which the ligation reaction occurs is about 38° C. In some embodiments, the temperature at which the ligation reaction occurs is about 39° C. In some embodiments, the temperature at which the ligation reaction occurs is about 40° C.

In some embodiments, the acceptor RNA and the donor RNA comprise at least two RNA nucleotides that have perfect complementarity. In some embodiments, the acceptor RNA and the donor RNA comprise 5 canonical base pairs in the lower stem. In some embodiments, the acceptor RNA and the donor RNA comprise 2 non-canonical base pairs in the lower stem. In some embodiments, the acceptor RNA and the donor RNA comprise 2 canonical base pairs in the upper stem. In some embodiments, the acceptor RNA and the donor RNA comprise 8 base pairs. In some embodiments, the base pairs are not contiguous. In some embodiments, the base pairs are contiguous.

Gene Editing Using gRNA

The synthetic gRNA described herein can be used with a suitable gene editing system for targeted gene editing which can result in a gene silencing event, or an alteration of the expression (e.g., an increase or a decrease) in the expression of a desired target gene. Accordingly, in some embodiments, the synthetic gRNA described herein can be used in a method for targeted transcription activation, targeted transcription repression, targeted epigenome modification, or targeted genome modification, the method comprising introducing into a eukaryotic cell: (a) a synthetic guide RNA (gRNA) as defined herein; (b) at least one CRISPR/Cas protein or a nucleic acid encoding the at least one CRISPR/Cas protein; wherein interactions between (a) and (b) and a target sequence in chromosomal DNA leads to targeted transcription activation, targeted transcription repression, targeted epigenome modification, or targeted genome modification.

In some embodiments, the synthetic RNA described herein can be used in a gene editing system comprising: the synthetic guide RNA described herein, wherein the RNA guide comprises a direct repeat sequence and a spacer sequence capable of hybridizing to a target nucleic acid; gene editing protein, and wherein the gene editing enzyme is capable of binding to the RNA guide and of causing a break in the target nucleic acid sequence complementary to the RNA guide.

In some embodiments, the synthetic RNA described herein can be used in a gene editing system comprising: the synthetic guide RNA described herein, wherein the RNA guide comprises a direct repeat sequence and a spacer sequence capable of hybridizing to a target nucleic acid; and a gene editing protein h; wherein the gene editing protein is fused to a deaminase, and wherein the gene editing protein fusion is capable of binding to the RNA guide and of editing the target nucleic acid sequence complementary to the RNA guide.

In some embodiments, the invention provides a method of altering expression of a target nucleic acid in a eukaryotic cell comprising: contacting the cell with a gene editing protein, and the synthetic guide RNA described herein, wherein the RNA guide comprises a direct repeat sequence and a spacer sequence capable of hybridizing to the target nucleic acid, and wherein the gene editing protein is capable of binding to the RNA guide and of causing a break in the target nucleic acid sequence complementary to the RNA guide.

In some embodiments, the invention provides a method of altering expression of a target nucleic acid in a eukaryotic cell comprising: contacting the cell with a gene editing protein, and the synthetic guide RNA described herein, wherein the RNA guide comprises a direct repeat sequence and a spacer sequence capable of hybridizing to the target nucleic acid, and wherein the gene editing protein is capable of binding to the RNA guide and editing the target nucleic acid sequence complementary to the RNA guide.

In some embodiments, the invention provides a method of modifying a target nucleic acid in a eukaryotic cell comprising: contacting the cell with a gene editing protein, and the synthetic guide RNA described herein, wherein the RNA guide comprises a direct repeat sequence and a spacer sequence capable of hybridizing to the target nucleic acid, and wherein the gene editing protein is capable of binding to the RNA guide and editing the target nucleic acid sequence complementary to the RNA guide.

In some embodiments, the gene editing method or system comprises a fusion protein with an effector that modifies target DNA in a site-specific manner, where the modifying activity includes methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein).

In some embodiments, the gene editing method or system comprises a fusion protein with enzymes that can edit DNA sequences by chemically modifying nucleotide bases, including deaminase enzymes that can modify adenosine or cytosine bases and function as site-specific base editors. For example, APOBEC1 cytidine deaminase, which usually uses RNA as a substrate, can be targeted to single-stranded and double-stranded DNA when it is fused to Cas9, converting cytidine to uridine directly, and TadA enzymes have been evolved to deaminate adenosine to inosine. Thus, ‘base editing’ using deaminases enables programmable conversion of one target DNA base into another. Various base editors are known in the art and can be used in the method and systems described herein. Exemplary base editors are described in, for example, Rees and Liu Nature Review Genetics, 2018, 19(12): 770-788, the contents of which are incorporated herein.

In some embodiments, base editing results in the introduction of stop codons to silence genes. In some embodiments, base editing results in altered protein function by altering amino acid sequences.

In some embodiments, the synthetic guide RNA described herein can be used in a gene editing method or system to modulate transcription of target DNA. In some embodiments, he synthetic guide RNA can be used in a gene editing method or system to modulate the expression of a target non-coding RNA, including tRNA, rRNA, snoRNA, siRNA, miRNA, and long ncRNA.

In some embodiments, the synthetic guide RNA described herein is used for targeted engineering of chromatin loop structures using a suitable gene editing system. Targeted engineering of chromatin loops between regulatory genomic regions provides a means to manipulate endogenous chromatin structures and enable the formation of new enhancer-promoter connections to overcome genetic deficiencies or inhibit aberrant enhancer-promoter connections.

In some embodiments, the synthetic guide RNA described herein is used in conjunction with a gene editing system for correction of pathogenic mutations by insertion of beneficial clinical variants or suppressor mutations.

### Therapeutic Applications

The synthetic guide RNA described herein can be used in a gene editing system for various therapeutic applications. Accordingly, in some embodiments, a method of treating a disorder or a disease in a subject in need thereof is provided, the method comprising administering to the subject a synthetic guide RNA described herein with a gene editing system. Various gene editing systems are known in the art and include for example CRISPR-Cas9, Cpf1, SpCas9, SaCas, Cas12, and prime editing Cas among others. The synthetic gRNA described herein can be used with any gene editing system.

In some embodiments, the synthetic guide RNA described herein can be used in conjunction with a gene editing system to treat various diseases and disorders, e.g., genetic disorders (e.g., monogenetic diseases), diseases that can be treated by nuclease activity, and various cancers, etc.

In some embodiments, the synthetic guide RNA described herein can be used in conjunction with a gene editing system to edit a target nucleic acid to modify the target nucleic acid (e.g., by inserting, deleting, or mutating one or more nucleic acid residues). For example, in some embodiments a CRISPR systems is used with the synthetic gRNA described herein and comprises an exogenous donor template nucleic acid (e.g., a DNA molecule or a RNA molecule), which comprises a desirable nucleic acid sequence. Upon resolution of a cleavage event induced with the CRISPR system, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the synthetic guide RNA described herein is used in conjunction with a gene editing system to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA).

In one aspect, the synthetic guide RNA described herein can be used in conjunction with a gene editing system for treating a disease caused by overexpression of RNAs, toxic RNAs, and/or mutated RNAs (e.g., splicing defects or truncations).

In some embodiments, the synthetic guide RNA described herein can be used in conjunction with a gene editing system to target trans-acting mutations affecting RNA-dependent functions that cause various diseases.

In some embodiments, the synthetic guide RNA described herein can be used in conjunction with a gene editing system to target mutations disrupting the cis-acting splicing codes that can cause splicing defects and diseases.

The synthetic guide RNA described herein can be used in conjunction with a gene editing system can for antiviral activity, in particular against RNA viruses. For example, to target viral RNAs using suitable synthetic RNA guides selected to target viral RNA sequences.

The synthetic guide RNA described herein can be used in conjunction with a gene editing system to treat a cancer in a subject (e.g., a human subject). For example, by targeting a RNA molecule that is aberrant (e.g., comprises a point mutation or are alternatively-spliced) and found in cancer cells to induce cell death in the cancer cells (e.g., via apoptosis).

The synthetic guide RNA described herein can be used in conjunction with a gene editing system to treat an infectious disease in a subject. For example, through targeting a RNA molecule expressed by an infectious agent (e.g., a bacteria, a virus, a parasite or a protozoan) in order to target and induce cell death in the infectious agent cell. The synthetic guide RNA described herein can be used in conjunction with a gene editing system to treat diseases where an intracellular infectious agent infects the cells of a host subject.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region.

Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%),

L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the invention, the DNA-targeting RNA and/or site-directed modifying polypeptide and/or donor polynucleotide are employed to modify cellular DNA in vivo, again for purposes such as gene therapy, e.g. to treat a disease or as an antiviral, antipathogenic, or anticancer therapeutic, for the production of genetically modified organisms in agriculture, or for biological research. In these in vivo embodiments, a DNA-targeting RNA and/or site-directed modifying polypeptide and/or donor polynucleotide are administered directly to the individual. A DNA-targeting RNA and/or site-directed modifying polypeptide and/or donor polynucleotide may be administered by any of a number of well-known methods in the art for the administration of peptides, small molecules and nucleic acids to a subject. A DNA-targeting RNA and/or site-directed modifying polypeptide and/or donor polynucleotide can be incorporated into a variety of formulations. More particularly, a DNA-targeting RNA and/or site-directed modifying polypeptide and/or donor polynucleotide of the present invention can be formulated into pharmaceutical compositions by combination with appropriate pharmaceutically acceptable carriers or diluents.

Pharmaceutical preparations are compositions that include one or more a DNA-targeting RNA and/or site-directed modifying polypeptide and/or donor polynucleotide present in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel.

Alternatively, drug delivery of therapeutics agents behind the BBB may be by local delivery, for example by intrathecal delivery.

Typically, an effective amount of a DNA-targeting RNA and/or site-directed modifying polypeptide and/or donor polynucleotide are provided. As discussed above with regard to ex vivo methods, an effective amount or effective dose of a DNA-targeting RNA and/or site-directed modifying polypeptide and/or donor polynucleotide in vivo is the amount to induce a 2 fold increase or more in the amount of recombination observed between two homologous sequences relative to a negative control, e.g. a cell contacted with an empty vector or irrelevant polypeptide. The amount of recombination may be measured by any convenient method, e.g. as described above and known in the art. The calculation of the effective amount or effective dose of a DNA-targeting RNA and/or site-directed modifying polypeptide and/or donor polynucleotide to be administered is within the skill of one of ordinary skill in the art, and will be routine to those persons skilled in the art. The final amount to be administered will be dependent upon the route of administration and upon the nature of the disorder or condition that is to be treated.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a DNA-targeting RNA and/or site-directed modifying polypeptide and/or donor polynucleotide may be obtained from a suitable commercial source. As a general proposition, the total pharmaceutically effective amount of the a DNA-targeting RNA and/or site-directed modifying polypeptide and/or donor polynucleotide administered parenterally per dose will be in a range that can be measured by a dose response curve.

Therapies based on a DNA-targeting RNA and/or site-directed modifying polypeptide and/or donor polynucleotides, i.e. preparations of a DNA-targeting RNA and/or site-directed modifying polypeptide and/or donor polynucleotide to be used for therapeutic administration, must be sterile. Sterility is readily accomplished by filtration through sterile filtration membranes (e.g., 0.2μηi membranes). Therapeutic compositions generally are placed into a container having a sterile access port, for example, an intravenous solution bag or vial having a stopper pierceable by a hypodermic injection needle. The therapies based on a DNA-targeting RNA and/or site-directed modifying polypeptide and/or donor polynucleotide may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The synthetic RNA described herein, along with a desired gene editing system components, can be delivered to a cell of interest by various delivery systems such as vectors, e.g., plasmids and delivery vectors.

The synthetic RNA described herein can be delivered by nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 1 (below).

TABLE 1
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 2 lists exemplary polymers for use in gene transfer and/or nanoparticle formulations.

TABLE 2
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 3 summarizes delivery methods for a polynucleotide encoding a Cas9 described herein.

TABLE 3

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system including the synthetic gRNA describe herein may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive the CRISPR system (e.g., including the synthetic gRNA described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, separate promoters drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A Cas9 and synthetic gRNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA or a Cas9-encoding mRNA, can be delivered in the form of RNA. Cas9 encoding mRNA can be generated using in vitro transcription. For example, Cas9 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas9 sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising gene editing system (e.g., including the synthetic gRNA described herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249: 1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the CRISPR system (e.g., including the Cas9 described herein) are provided as part of a pharmaceutical composition. In some embodiments, the pharmaceutical composition comprises any of the fusion proteins provided herein (e.g., including the nucleobase editor described herein comprising LubCas9). In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Kits

In one aspect, the synthetic gRNA described herein can be provided and or produced by a kits containing any one or more of the elements disclosed in the above methods and compositions. For example, a kit may include an acceptor RNA, a donor RNA, a ligase, and suitable buffering reagents. The acceptor RNA, donor RNA and ligase may be any that are disclosed herein.

In some embodiments, the kit further comprises a nucleobase editor.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. Traditional Synthesis of RNA

Traditional synthesis of RNA includes the use of plasmid DNA and solid-phase synthesis using phosphoramidite chemistry (“synthetic RNA”). A comparison of chemical synthesis and enzymatic-based synthesis of synthetic RNA is detailed below.

Directionality

Synthetic RNA is typically synthesized in the 3′ to 5′ direction. For sgRNA, this means that most side products are those with truncations in the spacer region at the 5′ terminus which will lead to lower on-target editing.

Substrates

Chemical synthesis utilizes highly reactive monomers. These monomers are chemically protected by functional groups to decrease side reactions and ensure that the desired reactions happen at the correct stage of synthesis. These monomers are referred to as “amidites”, referring to the phosphoramidite functional group common among them. The chemical groups surrounding the phosphoramidite core can be heavily modified and need not resemble naturally occurring nucleotides. For this reason, chemical synthesis can be used to install highly modified monomers including modified sugars, bases, backbones or functional groups that do not resemble natural nucleotides.

Sequentially

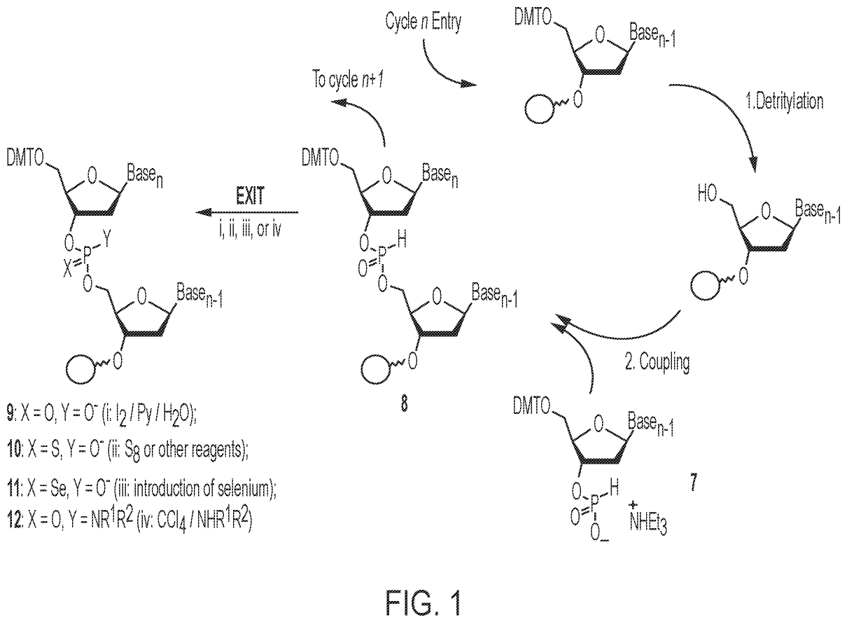

Synthetic RNA is typically synthesized through sequence-controlled polymerization on a solid support. Chemical synthesis is performed in cycles, each comprising various steps (see FIG. 1). This sequence is designed to, as much as possible, prevent insertions of undesired nucleotides or deletions. Oligos that fail to become incorporated into the growing polymer at any given stage are chemically “capped” to prevent them from extending beyond the position in the sequence in which they “failed” to incorporate. “Coupling efficiency” is the term that refers to overall efficiency of each cycle. This value is largely dependent on the nature of the amidite but can also be impacted by the design of the instrument or scale of the reaction. Typical DNA coupling efficiencies are on the order of 98-99.5% and are generally greater for DNA than RNA.

Purification

Oligo products are first deprotected and cleaved from the solid support before purification. Purification is typically performed by either electrophoretic separation (ie. polyacrylamide gel electrophoresis or “PAGE”) or, more commonly, column chromatography (i.e., HPLC). HPLC is performed using a stationary phase of either anion-exchange or reverse-phase ion-pairing media. Both methods exponentially lose resolution as the length of the full-length product increases. This is especially problematic as the most common side products in the mixture with the FLP after purification will be similar in length to the full-length product. Furthermore, the large-scale synthesis required for GMP-grade material typically features lower coupling efficiencies, resulting in decreases in purity and increases in the number of addition products relative to the more commonly used small scale syntheses used to produce material for research purposes. The differences in coupling efficiencies most commonly result from the requirement for longer coupling times as synthetic scales increase. Oligos of lengths around 100 nt, (i.e., guide RNAs used in base editing) are difficult to physically separate from those that are only a few nt shorter. Due to these limitations, purities of gRNAs from CMOs are most often obtained in the 50-90% range. For typical synthetic gRNA synthesis strategies, most of the impurities comprising the remaining 10-50% contain deletions in the spacer region (primarily truncation products) and addition products. These types of impurities will result in poor editing efficiencies and/or off target editing.

Advantages of a Modified Process for Production of Long RNAs

A method to produce long RNAs (e.g., 100 nucleotides or more) of high purity by chemical synthesis is desirable for several reasons, including: reduced off-target editing, high efficiency editing, increased purity in comparison to traditional synthesis approaches, increased yield, reduced cost, and versatility for modification of nucleotides in the synthesized RNA.

A modified chemical synthesis approach to produce RNAs can result in reduced off-target editing. Purity and off-target editing are likely correlated. There is evidence to suggest the opposite for truncation products (the major side product) which appear to decrease both off-target and on-target edits; however, addition products would likely increase off-target edits.

A modified chemical synthesis approach to produce RNAs can result in high efficiency editing. This is at least because most impurities (e.g., truncations) decrease the activity of editing.

A modified chemical synthesis approach to produce RNAs can result in increased purity in comparison to traditional synthesis approaches. The increased purity of the synthetic RNAs would be more amendable to regulatory agency approval for use in treating human patients.

A modified chemical synthesis approach to produce RNAs can result in increased yield and decreased cost. The yield of a synthetic RNA process typically decreases exponentially with the length of the synthetic RNA. Typically, only 3-5% of the theoretical yield is obtained after purification, although 20-30% full-length product (FLP) is made in the reaction (thus, >90% of the FLP made in the reaction is lost during purification). As an example, the cost may be $1-2 million for 5 grams of GMP-grade FLP (10 grams of material at 50% purity). If the majority of FLP can be isolated during purification, then the cost of production will decrease 5-10 fold and be associated with an increase in purity.

Lastly, a modified chemical synthesis approach to produce RNAs can allow for the ability to specifically install modified nucleotides and chemical functionalities which are not possible using enzymatic synthesis.

### Example 2: Ligation-Based Approach to Synthesis of RNA

The ligation-based approach described in this example makes use of one or more RNA fragments that are subsequently ligated to create a full-length guide RNA (gRNA). Creation of the one or more RNA fragments allows for a greater post-purified yield of the gRNA because of better separation of side-products, among other things.

One aspect of the ligation-based approach uses a helix formed between the crRNA and the tracrRNA molecules that make up the dual-guide RNA system used in biology (known as the repeat-anti-repeat helix) to template the enzymatic ligation of two synthetic RNAs. This is illustrated in FIG. 2.

In some embodiments, the length and sequence composition of this helix is modified to promote proper non-covalent assembly and to create optimal ligation sites for enzymes compatible with RNA ligation. Nucleotide lengths from 5 to 50 are desirable for this type of association. In some embodiments, enzymatic ligations may be more efficient when the donor nucleobase is a C and the acceptor is an A. In some embodiments the Tm (melting temperature) of the non-covalently assembled RNAs is greater than 0° C., 1° C., 2° C., 3° C., 4° C., 5° C., 6° C., 7° C., 8° C., 9° C., 10° C., 11° C., 12° C., 12° C., 14° C., 15° C., 16° C., 17° C., 18° C., 19° C., 20° C., or more. For example, the length of the stem can be modified to be long enough to promote formation of the stem loop above the temperature at which ligation will be performed and also to avoid non-ligation compatible self-structures. As a further example, the variability of the spacer sequence could lead to base pairing that is not compatible with ligation, this can be avoided by adding an oligonucleotide with a sequence complementary to the spacer sequence prior to combining with the donor sequence.

In some embodiments, the RNA comprising the tracrRNA sequences are synthesized with a phosphate at the 5′-terminus (termed “donor”) which is ligated to the 3′-terminus of a second RNA which comprises the variable protospacer region (termed “acceptor”) by one of multiple ligases. In some embodiments, the RNA comprising the tracrRNA sequences are synthesized such that a portion of the tracrRNA contains a phosphate at the 5′-terminus.

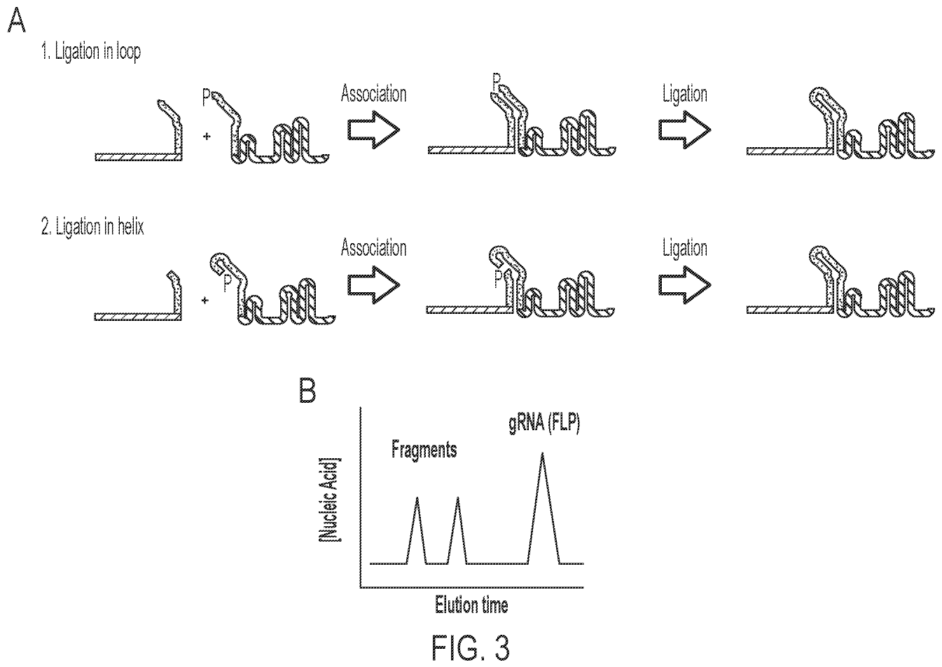

Two forms of ligation are possible with this approach (FIG. 3, panel A (1) and (2)), both of which are found within the stem loop region. The first form of ligation occurs within the terminal loop of the hairpin, which is a natural site of T4 RNA Ligase 1. The second form of ligation occurs within the duplex which is a natural of T4 RNA Ligase 2 and DNA ligases. One of the advantages of this form of ligation is that fragment impurities are readily removable because of the marked differences in elution time between the fused gRNA and the fragment impurities (FIG. 3, panel B).

Another ligation-based approach of the invention involves the ligation of two or more RNA fragments via a non-templated approach. In this approach, the 3′ hydroxyl of the 3′ terminal end of the donor molecule is chemically blocked or removed (e.g., dideoxynucleotide) and an enzyme (e.g., T4 RNA Ligase 1) would catalyze proper ligation between the two molecules. Generally, this ligation strategy is favored at higher concentration.

The ligation approach described in this example makes use of ligases, a class of enzymes that combine sections of nucleic acids with each other. An advantage of using such ligases is that the resulting linkages between the fragments of RNA are indistinguishable from naturally occurring RNA or DNA. The ligases function on RNA or DNA and perform reactions with high efficiency.

In some embodiments, the RNA fragments meant for ligation can be brought into physical proximity for the ligation reaction by use of a nucleic acid template that has complementarity to a first and a second RNA fragment. This template is referred to herein as a splint. Splints can be designed with imperfect pairing to generate loops that are amenable to ligation by a particular ligase, e.g., T4 RNA Ligase 1. In some embodiments, splints are used to bring together more than two fragments of RNA.

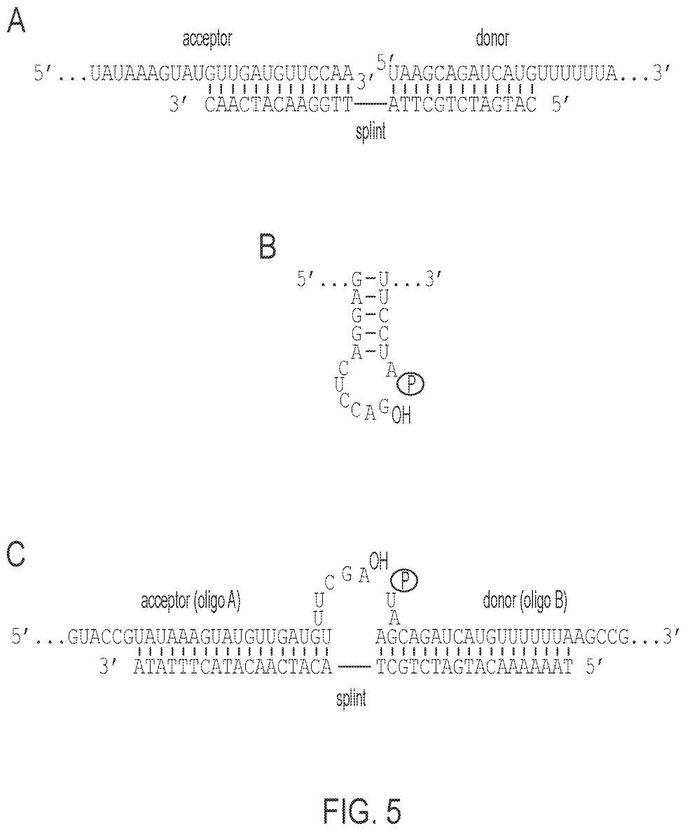

In some embodiments, the RNA fragments can be associated by base-pairing with each other before the ligation reaction. This approach is referred to herein as “self-templating.” Using the self-templating approach, the locations of the stem loop that can be selected for ligation of the RNA fragments include the loop or in the helix where one of the oligos contains a short stem-loop (e.g., self-templating nick). Nicks can be included on splints, overhangs, blunt ends, and bulges can also be used (see FIG. 5).

In some embodiments, the ligation reaction can proceed with high yield without the need for physical association of the RNA sections beforehand. In view of this, select ligation reactions do not require the use of a splint or self-templating.

Different ligation approaches of the invention are depicted in FIG. 5.

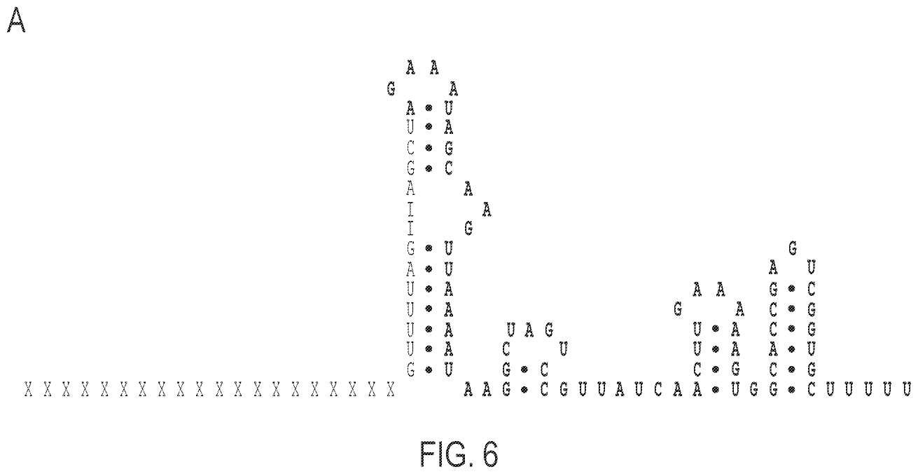

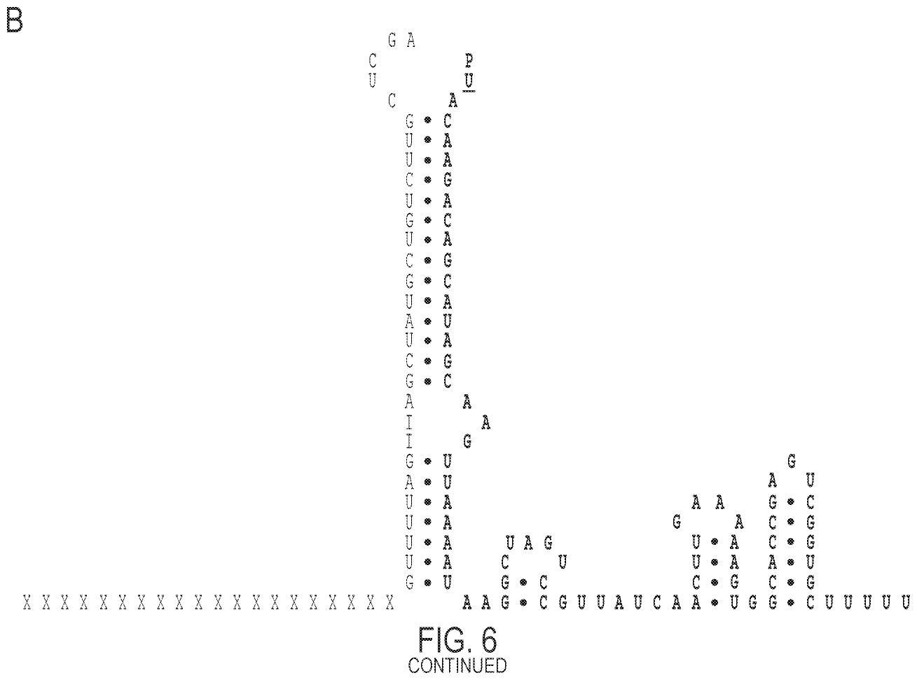

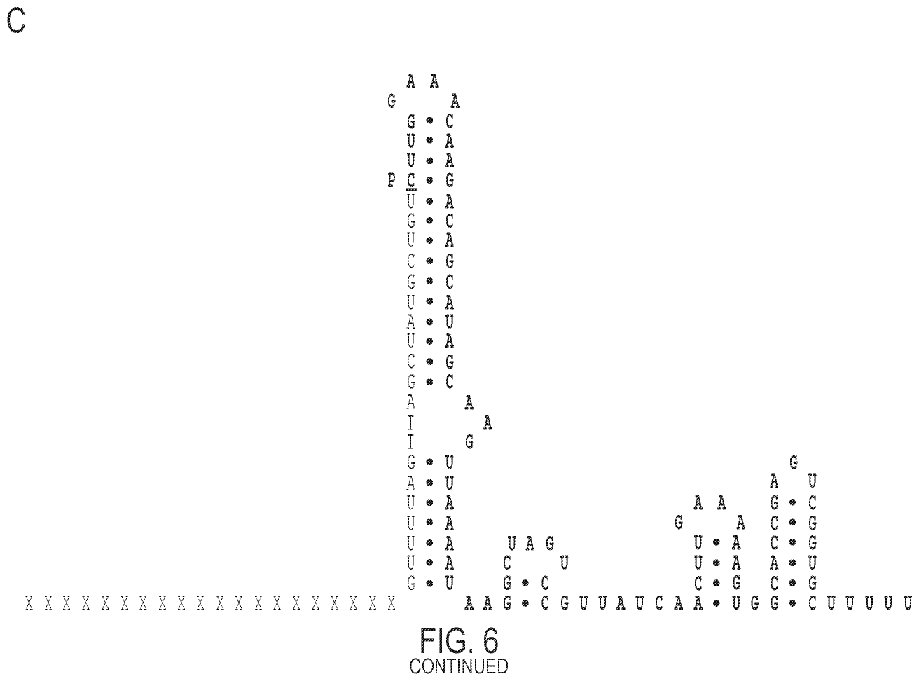

Various ligation designs are being examined using the ligation-based approaches described herein (FIG. 6). As depicted in FIG. 6, one of these designs involves the ligation of two RNA fragments at the loop of the stem loop (FIG. 6, panel B); another design involves the ligation at the helix of the stem loop (FIG. 6, panel C).

These ligation strategies are different from other reported chemical ligation strategies used to synthesize sgRNA as the described ligation strategies form a natural phosphate linkage at the site of ligation. The advantage of using a segmented synthetic approach is that short sections of RNA can be produced with greater purity post-purification compared to full length sgRNA. In some embodiments, the 5′ acceptor is the smallest RNA fragment (30-50 nucleotides) and can thus be purified to a high level before ligation. The 3′ donor is terminated with a phosphate that is required for synthesis and thus only the full-length fragment will be incorporated into the full-length product (i.e., truncations are not substrates).

This advantage is increased when considering gRNAs that are greater than 100 nucleotides, such as pegRNA or Cas12b gRNA. The types of enzymatic ligations are very high yielding (>80%) and the oligonucleotide starting material can be separated from ligated product with high selectivity, ensuring that full-length product is very pure. Furthermore, these types of enzymatic ligations are relatively inexpensive and scale well.

### Example 3: Exemplary Ligation-Based RNA Synthesis Protocol

An exemplary protocol for the synthesis of synthetic RNA is provided below.
1. Stem size and ligation site (loop or helix) are selected based on i) requirements for the natural substrate of the ligase used (e.g., loop vs helix design) and ii) affinity of the bimolecular helix which is determined using of thermodynamic algorithms for RNA duplex stability.
2. RNA fragments are synthesized using standard phosphoramidite chemistry. The 3′ RNA fragment (donor) contains a terminal 5′ phosphate that is included in the last step of synthesis.
3. RNA fragments are purified by HPLC (fragments can also be purified by using either anion exchange chromatography (AEX) or with ion-pair reversed-phase chromatography (IP-RP).
4. Annealing: Combine each oligonucleotide (0.01-1 mM) with annealing buffer (25 mM KCl, 0.025 mM EDTA). Heat to 80° C. for 0.5-5 min then cool at 0.1° C./sec until 25° C.
5. Ligation: RNA ligase buffer is added to achieve a 1× concentration (50 mM Tris-HCl, 10 mM MgCl2, 1 mM DTT, 1 mM ATP, pH 7.5 at a temperature between 20-37° C.). Add 5-10 U of T4 RNA ligase 1/nmol phosphorylated 5′ ends. Incubate overnight at a temperature between 20-37° C. Stop by addition of 0.5 M EDTA.
6. Purification by ion-pair reversed-phase chromatography (IP-RP) (potentially also AEX) is be used.
7. Analysis by 6% PAGE-D gel stained with SYBR Safe and IP-RP HPLC.

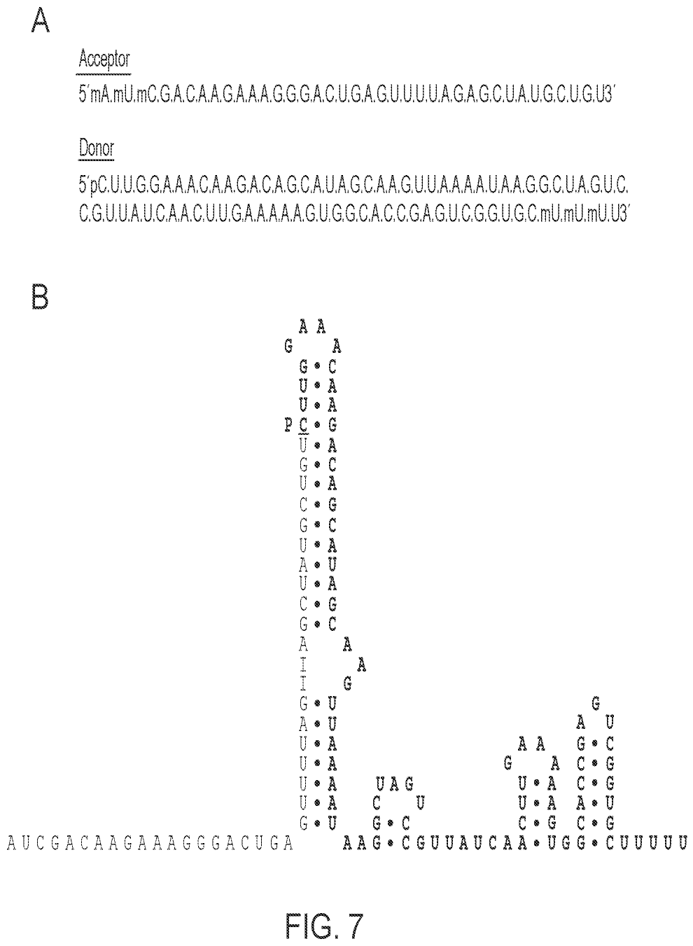

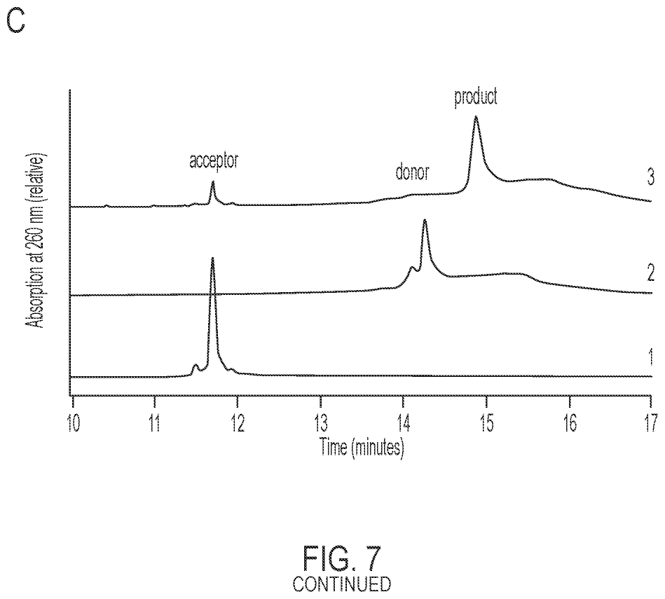

Exemplary results of a ligation experiment are presented in FIG. 7. For this ligation, the reaction contained 10 μM donor fragment, 10 μM acceptor fragment, lx T4 RNA Ligase 2 Reaction Buffer (NEB), and 20 units of T4 RNA Ligase 2, and was performed at 37° C. FIG. 7, panel A shows the sequences used for the ligation experiment. The results of the stem-ligation is shown in FIG. 7, panel C. Full-length product was detected by HPLC, as well as separation of the RNA acceptor and donor fragments from the full-length product.

### Example 4: Differences Between Described Approach and Prior Methods

The ligation approaches of the present invention differ from previously described RNA ligation approaches because, among other things, the previously described ligation approaches have relied upon non-natural linkages between fragment RNA molecules and/or used a non-templated ligation approach such as through the use of azide-alkyne cycloaddition reactions to couple smaller RNA molecules into sgRNAs via non-natural tirazole linkages. The use of non-natural linkages as previously described have several disadvantages, including the possibility that the non-natural linkages may have undesirable effects in biological systems.

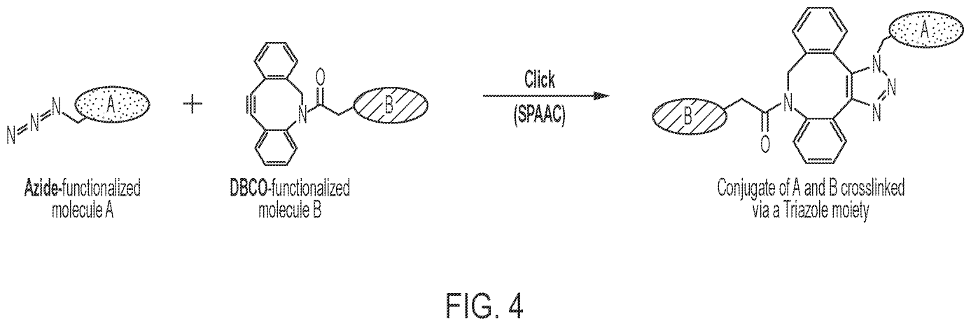

The ligation approaches described herein also differ from previously used chemical ligation strategies that use other versions of “click-chemistry” or other chemical bioconjugation methods to combine RNA fragments into full-length sgRNA (see FIG. 4). Other previously described approaches to combine RNA fragments include the use of amide-ligation chemistry (e.g., coupling of 18 atom linker by amides) and self-templating to form sgRNAs. The prior approaches are disadvantageous at least because: i) the chemical groups used for ligation are not likely incorporated at high yield, unlike the incorporation of a phosphate in the invention described herein; and ii) the linkages previously used are non-natural and significantly larger than the natural phosphodiester linkage. Because of these limitations, the previously described RNA ligation approaches may compromise potency and introduce additional regulatory burdens.

### Example 5: Production of sgRNA— Comparison of Ligases

In this example, two constructs, one in which ligation occurs in the loop and the other in which ligation occurs in the stem (FIG. 3), were evaluated to determine which ligase resulted in the highest yield of sgRNA product. T4 RNA Ligase 1 was used for ligation at the loop, and T4 RNA Ligase 2 was used for ligation in the stem.

FIG. 3, panel A, depicts two locations for enzymatic ligation that were evaluated: (i) in the loop of the stem-loop, and (ii) in the helix. In both cases this stem-loop was extended and used to associate the sections for enzymatic ligation. FIG. 3, panel B, depicts a representative graph that shows after ligation a final purification step can be performed comprising HPLC to remove RNA fragments that did not ligate. Purification of the RNA fragments from the full-length product is possible.

A process was developed to synthesize single-guide RNA using a combined chemical and enzymatic strategy that overcomes challenges that limit the purity and final (post-purified) yield of synthetic RNAs. This approach, termed L.O.N.G.E.S.T. (ligation of nucleic acid guides using enzymes and self-templating), uses a ligation-based approach where two or more partially complementary synthetic RNAs are ligated using an enzyme. In one embodiment, a helix is formed between crRNA and tracrRNA molecules that make up a dual guide RNA system used by SpCas9 in biology (known as the repeat-anti-repeat helix) to template the enzymatic ligation of two synthetic RNAs (FIG. 3). The length and sequence composition of this helix can be modified to promote proper non-covalent assembly and to create optimal ligation sites for enzymes compatible with RNA ligation without decreasing the activity of the RNP complex. The RNA comprising most of the tracrRNA sequences can be synthesized with a phosphate at the 5′-terminus (termed donor) which is ligated to the 3′-terminus of a second RNA which comprises the variable protospacer region (termed acceptor) by either T4 RNA Ligase 1 or T4 RNA Ligase 2. Two forms of ligation are exemplified with this approach (FIG. 3), first within the terminal loop of the hairpin (substrate of T4 RNA Ligase 1) and, second, within the duplex (substrate of T4 RNA Ligase 2 and DNA ligases).

In these experiments, both T4 RNA Ligase 1 and T4 RNA Ligase 2 were evaluated, as well as donor/acceptor RNA fragment designs. The protospacer for these gRNAs was of Alpha 1. It was determined that T4 RNA Ligase 2 produced the highest yield and minimal side products as compared to T4 RNA Ligase 1. It was also found that a donor/acceptor design that, compared to “standard” gRNA designs, adds only two bases to the final sgRNA product and produces, under the conditions examined here, quantitative yield of sgRNA.

Experimental Conditions.

All reactions that contained T4 RNA Ligase 2 contained 10 μM donor, 10 μM acceptor, lx T4 RNA Ligase 2 reaction buffer, and 20 units of T4 RNA Ligase 2. All reactions were performed for 15 hrs at 37° C.

For reactions with T4 RNA Ligase 1, all reactions contained 10 μM donor, 10 μM acceptor, NEB reaction buffer, 1 mM ATP, and 20 units of T4 RNA Ligase 1. Some reactions also contained 25% (wt/vol) PEG 8000. Reactions were performed for 15 hours at 25° C.

All reactions were performed at 50 μL in a thermocycler. To form the pre-ligation complex, solutions were first heated to 70° C. with all components except the ligase and slow cooled at 0.1° C./s to either 37° C. or 25° C., for T4 RNA Ligase 2 or T4 RNA Ligase 1, respectively.

RNA Donor and RNA Acceptor Designs—T4 RNA Ligase 1 and T4 RNA Ligase 2

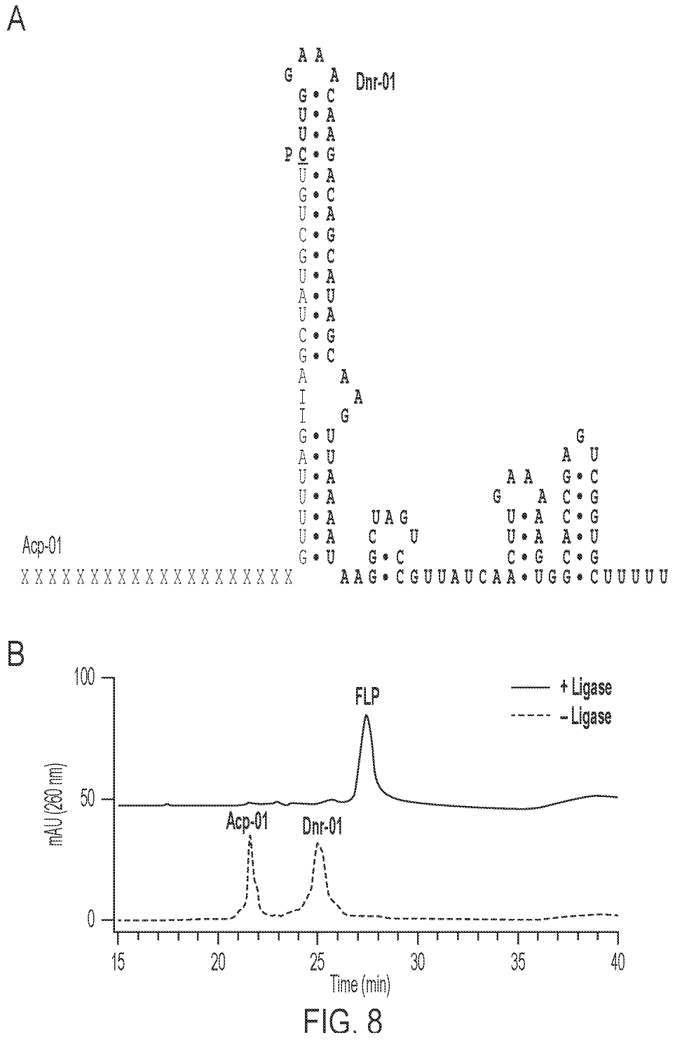

The stem-ligation design that consisted of RNA acceptor 1 (Acp-01) and RNA donor 1 (Dnr-1) was first evaluated. The post-ligation helix of the tetraloop contained 14 base pairs total (10 base pairs between fragments in the pre-ligation complex) in the upper helix with mixed GCAU content (FIG. 8, panel A). This reaction was high yielding with little to no detectable amounts of fragments in samples where T4 RNA Ligase 2 is present (FIG. 8, panel B). Control reactions (not shown) with ligase and only Dnr-01 or Acp-01 did not show formation of side reactions.

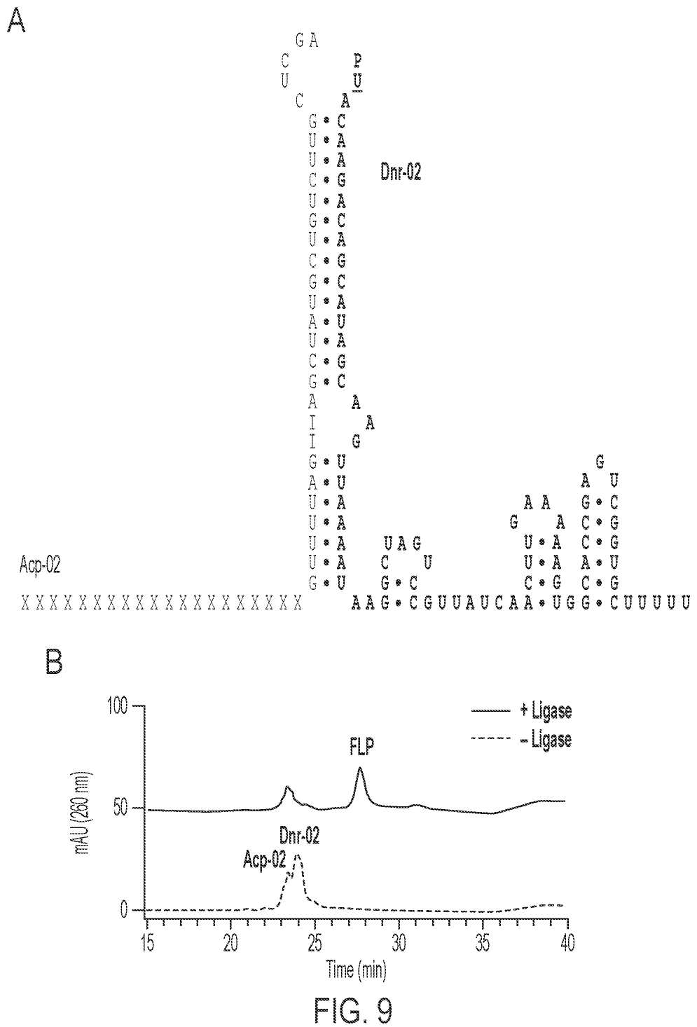

Another RNA donor and RNA acceptor design that was evaluated was the helix-ligation design of RNA acceptor 2 (Acp-02) and RNA donor 2 (Dnr-2). The post-ligation helix contained 14 base pairs in the upper helix with mixed GCAU content (FIG. 9, panels A and B). This reaction was lower yielding (˜60%) than reactions with T4 RNA Ligase 2. Control reactions (not shown) with ligase and (phosphorylated) Dnr-02 only showed formation of side reactions (cyclization of Dnr-02 is possible with T4 RNA Ligase 1). Reactions between Acp-02 and Dnr-02 in the presence of T4 RNA Ligase 2 did not form product as T4 RNA Ligase 2 requires a double stranded complex. The data from these experiments suggests that T4 RNA Ligase 2 is desirable over T4 RNA ligase 1. The remaining data were generated using T4 RNA ligase 2.

RNA Donor and RNA Acceptor Designs—Effect of GC Content and Stem Nucleotide Length

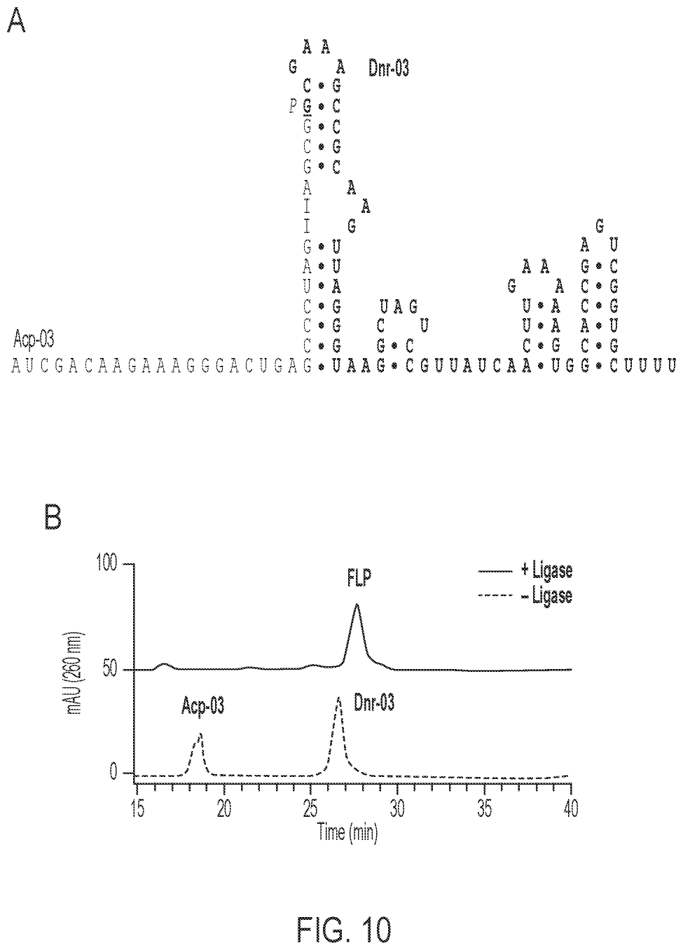

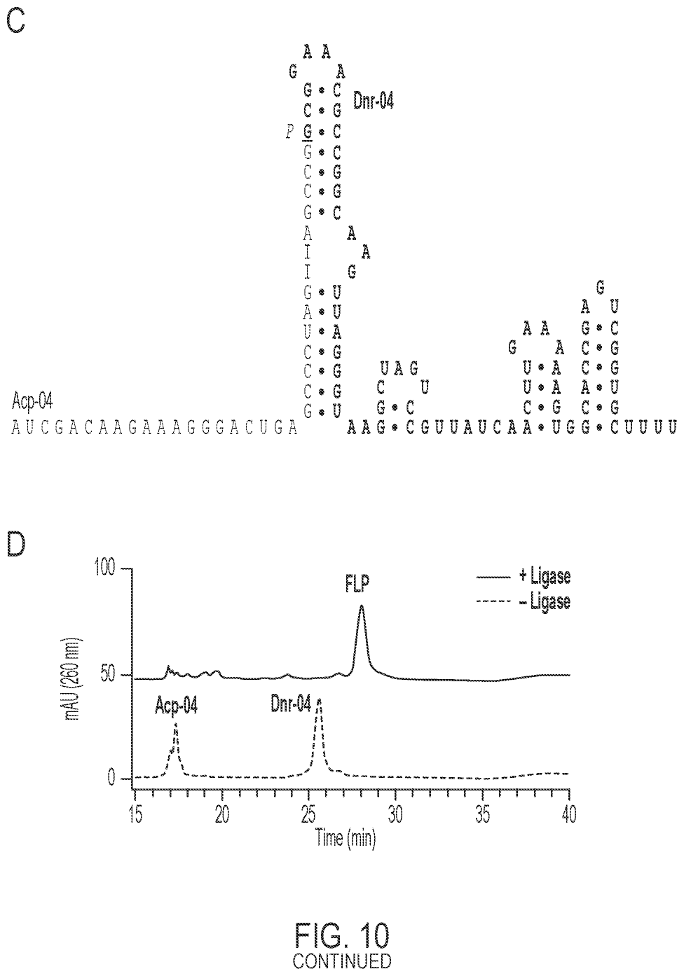

Two RNA constructs were evaluated that shared the following characteristics as compared to the initial Dnr-01/Acp-01 tetraloop design: i) higher GC content in the upper and lower stem, and ii) a shorter upper stem (FIG. 10, panels A-D). While the lower stem is understood to interact with Cas9, previous reports have shown that three of the four bases in the U track can be replaced by GC base pairs. sgRNAs with these substitutions were evaluated and it was found that, while active, their activity is less than that of standard sgRNA designs.

The data showed that reactions between Acp-03 and Dnr-03 with ligase is productive and is compatible with high yielding synthesis. Reactions between Acp-04 and Dnr-04 with ligase were productive, appearing to go to completion as indicated by the loss of the Dnr-04 peak. The Acp/Dnr-04 system represents a significant advance over Acp/Dnr-01 system as it is 7 base pairs shorter but still as productive.

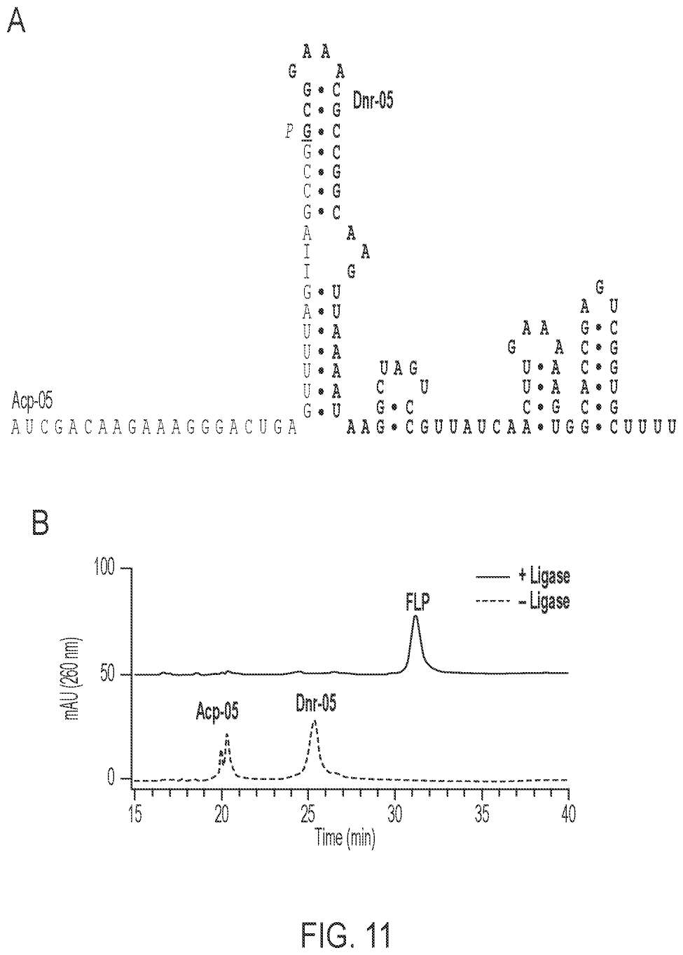

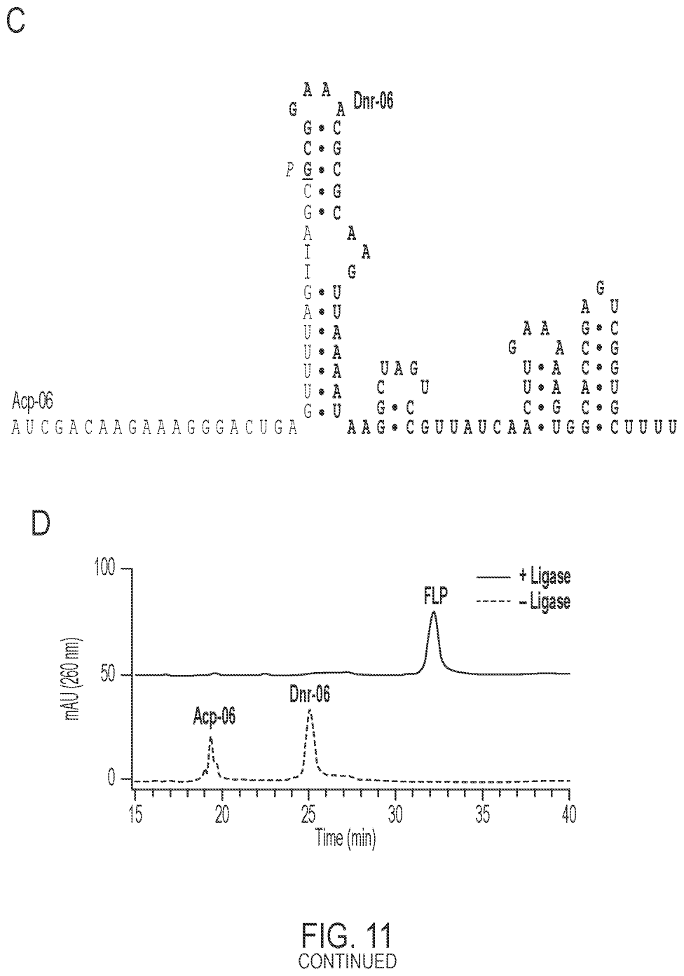

After decreased editing results from a study using gRNAs that had the U track of the bottom stem partially replaced with GC base pairs (as shown for Acp/Dnr-03 and Acp/Dnr-04), designs were evaluated that did not change the U track of the bottom stem and also included a shorter upper stem (FIG. 11). A design (Acp/Dnr-06) was also examined that had an analogous upper stem sequence to that of the sgRNA formed from ligation of Acp/Dnr-03, however, the ligation site was placed one bp further away from the tetraloop.

The data showed that reactions between Acp-05 and Dnr-05 with ligase were as productive as the Acp/Dnr-04 reaction system—indicating that the higher GC content in the lower stem loop in these earlier designs was not required for quantitative reactions (at least with the Alpha-1 protospacer). Reactions between Acp-06 and Dnr-06 with ligase was also productive. Because the upper stem loop was analogous to that of Acp/Dnr-03 with only the position of the ligation site changing, these results indicate that the ligation site at least three base pairs away from the tetraloop allows for efficient ligation. The Acp/Dnr-06 system demonstrated that minimal changes to the “standard” sgRNA design are compatible with high yielding synthesis as it only possesses a single additional base pair.

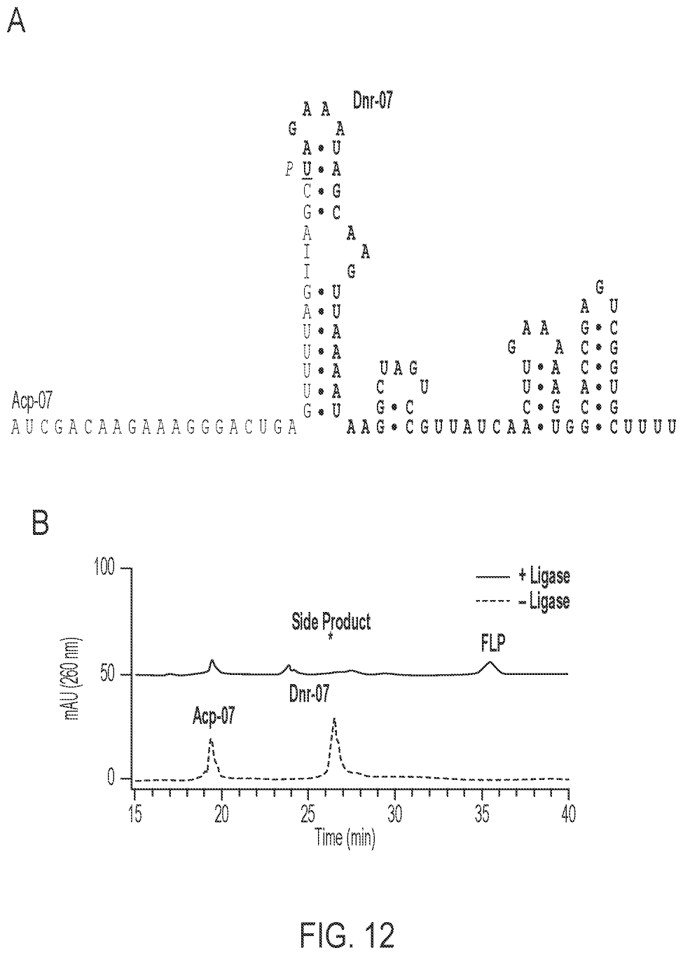

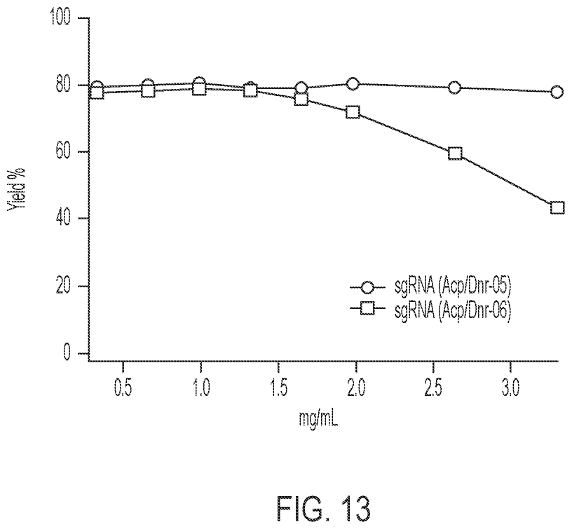

A “standard” sgRNA design was examined to see if it was compatible with ligation (FIG. 12). Because the upper stem loop contains only four base pairs the ligation site was included just two base pairs away from the tetraloop. Reactions between Acp-07 and Dnr-07 in the presence of the ligase were low yielding (FIG. 13). Additional side reactions were also present, further indicating that a well assembled duplex is useful for a high yielding reaction.

RNA ligation reactions were also performed using the same donor fragment (Dnr-05) with two different acceptor fragments (Acp-05 and Acp-05_v2) that varied only in the protospacer sequence that is not required for self-assembly between the donor and acceptor fragments. This illustrates the concept of using a universal donor in combination with various acceptor fragments.

RNA Donor and RNA Acceptor Designs—Effect of RNA Concentration on Productivity of Reaction

Studies were conducted to evaluate the effect of RNA concentration on the productivity of reaction. For these studies, Acp/Dnr-05 and Acp/Dnr-6 were used. Acp/Dnr-05 has a more stable acceptor/donor duplex in comparison to Acp/Dnr-6. In these studies, the concentration of both fragments were evaluated as it related to the productivity of the sgRNA productivity (FIG. 13). The data show that having a more stable A/D duplex enables higher yield at higher concentration of substrate.

Based on these data, 1 mg/ml or higher can be a suitable concentration for manufacturing. Also, temperature of the ligation reaction will also impact the data (note that all experiments described here were performed at 37° C.). It has also been shown that T4 RNA ligase 2 is effective at 20° C. FIG. 13 shows that the yield maxes out at approximately 80% but this may be because there are side products like truncations from the donor fragment that are not compatible with ligation but still add to the absorption of starting material and thus this value is underestimated.

Thermodynamic Effects on sgRNA Productivity

Under the conditions that were tested, a more thermodynamically stable duplex (formed by increasing length and/or GC content) provided higher yield up to a point but these changes to the “standard” design are not required to form product as still 60% was obtained while only modest modifications can enable much higher yield, this advantage does not scale linearly as it approaches a limit at just 1 extra bp—as can be seen from the result that both 1 and 10 extra bps provides a similar yield while the yield of the “standard” sgRNA design with 4 bps in the upper helix is significantly lower than if just 1 additional bp is used in the upper helix (AD-06).

Of note is that all of the studies described here were performed at 37° C. and this T4 RNA ligase 2 may tolerate lower temperatures as well, and, if so, a higher yield with the “standard” 4 bp upper helix at lower temperatures is likely possible. Thus, changing the conditions results in changes to the energetic off assembly. Thus various parameters, including external or intrinsic to the RNA design, can be used to obtain this objective. These conditions include, for example, changes to the reaction temperature and to the concentration of the RNA.

Conclusion

These data established design rules for fragments and ligases to use for the L.O.N.G.E.S.T. approach. In particular, it was found that T4 RNA Ligase 2 is higher yielding than T4 RNA Ligase 1 and produces fewer side reactions. It was also found that T4 RNA Ligase 2 accommodates double stranded substrates with ligation sites that are three or more base pairs away from the tetraloop. It was also found a fragment design (Acp/Dnr-06) that is high yielding and only contains one additional base pair compared to that of the standard sgRNA design (102 nucleotides vs. 100 nucleotides, respectively). Further studies will be aimed at evaluating the Acp/Dnr-06 design with respect to editing activity and scaling up reactions with this system.

### Example 6: Tolerance to Backbone Modification and Temperature Tolerance

Tolerance to Backbone Modification

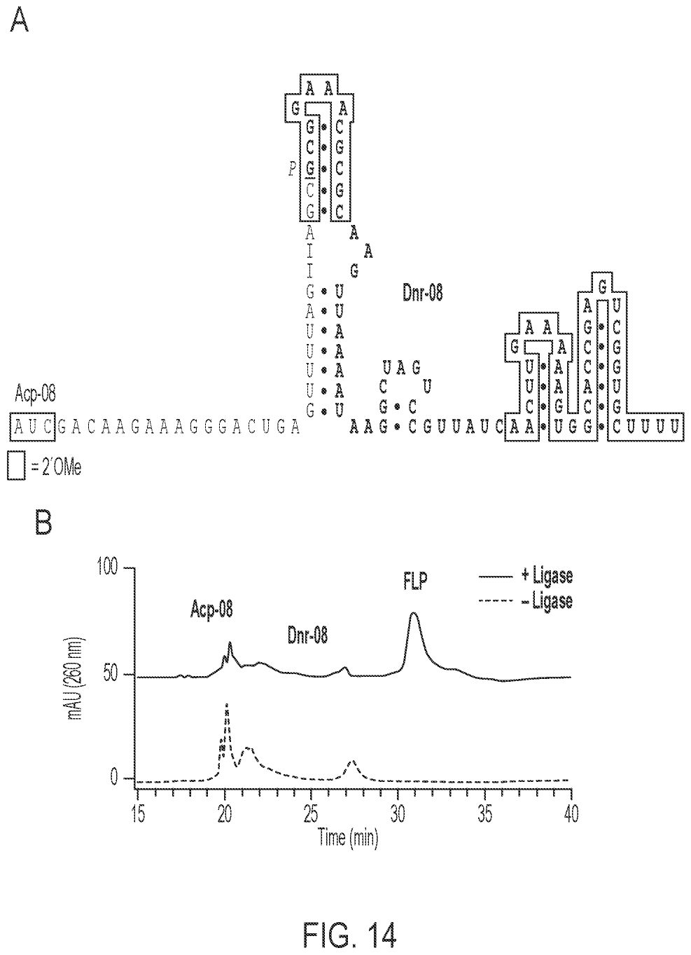

Various fragments have been analyzed to determine tolerance to backbone modification. The following fragments have been analyzed: i) fully RNA, ii) those that contain 2′ O-methyl and phosphorothioate groups at the termini, typically referred to as “end mods,” and iii) sequences that contain 48 nucleotides (48%) modified with 2′ O-methyl groups including modifications at the site of ligation (both the 5′ donor nucleotide and the 3′ acceptor nucleotide). Two sets of modified guides have been tested that are based on the Acp/Dnr-05 and Acp/Dnr-6 designs (AD_09 and AD_08, respectively). Data from these studies are shown in FIG. 14, panels A and B. These data collectively show successful reactions of extensively modified fragments.

Tolerance to Temperature

sgRNA were produced at either 20° C. and 37° C. The data from these experiments showed that reactions at either of the tested temperatures were productive. The finding that the reaction is also functional at 20° C. (room temperature) shows that the reaction is robust and can work at a temperature that will make manufacturing easier.

### Example 7: Base Editing in Cells

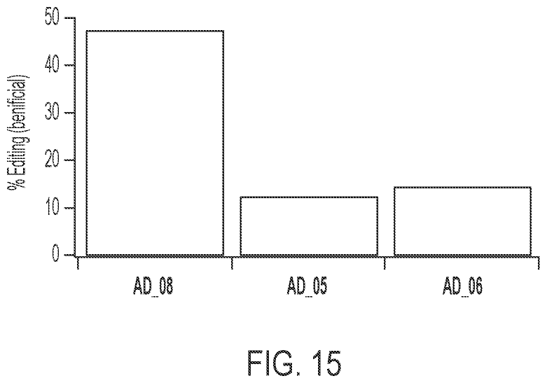

Base editing activity against specific gene targets for the purified products was successfully performed in mammalian cells. These data were obtained using DA-05, DA-06 and AD-08 from reactions of Acp-05 and Dnr-05, Acp-06 and Dnr-06, and Acp-08 and Dnr-08, respectively. (FIG. 15). For these studies, a target site in fibroblast cells were edited using an adenine base editor (ABE) and one of three guide RNAs (AD-08, AD-05, AD-06) synthesized using self-templating ligation method.

### Example 8: Cas12b Guide RNA

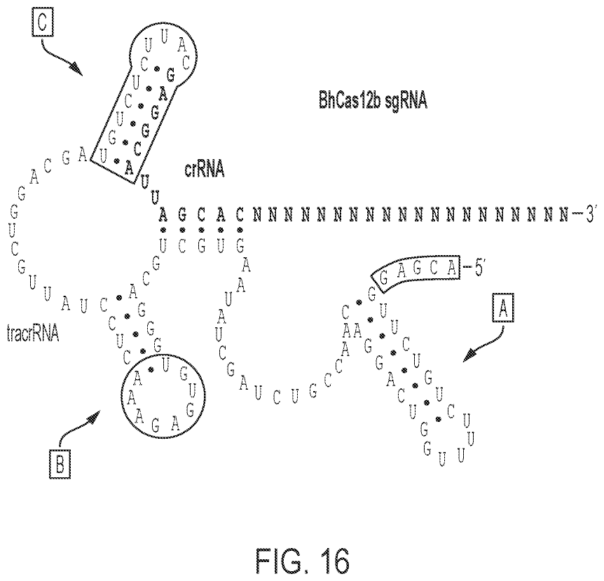

The methods disclosed herein can be used to synthesize Cas12b sgRNA. FIG. 16 is a schematic that depicts a sequence and configuration associated with an exemplary Cas12b sgRNA from Bacillus hisashii, bhCas12b sgRNA. Various features, including various secondary structures such as tetraloops, can be used as targets for splitting and ligating to create desired Cas12b sgRNA.

FIG. 16 shows various exemplary positions that can be targeted to split the sgRNA followed by subsequent ligation according to the methods described herein. FIG. 16 shows the secondary structure of bhCas12b sgRNA, which contains both a variable protospacer region and invariable region. Labels A, B, and C indicate hairpin loop structures that can be targeted as positions to split the sgRNA. The duplex of hairpin labeled as C can be extended at its loop-proximal position to promote donor and acceptor hybridization as this tetraloop does not contact the Cas protein.

Sequences

Table 4 (below) shows sequences referenced in the Examples and in the corresponding Figures.

TABLE 4
Referenced Sequences

| Fragment name | Sequence |
|---|---|
| Acp-0l | mA*mU*mC*GACAAGAAAGGGACUGAGUUUUAGAGCUAUGCUGU |
| | (SEQ ID NO: 6) |
| Dnr-01 | pCUUGGAAACAAGACAGCAUAGCAAGUUAAAAUAAGGCUAGUCCGU |
| | UAUCAACUUGAAAAAGUGGCACCGAGUCGGUGCmU*mU*mU*U |
| | (SEQ ID NO: 7) |
| Acpt-01 | mA*mU*mC*GACAAGAAAGGGACUGAGUUUUAGAGCUAUGCUGUCU |
| | UGCUCGA (SEQ ID NO: 8) |
| Dnr-02 | pUACAAGACAGCAUAGCAAGUUAAAAUAAGGCUAGUCCGUUAUCAA |
| | CUUGAAAAAGUGGCACCGAGUCGGUGCmU*mU*mU*U (SEQ ID |
| | NO: 9) |
| Acp-03 | mA*mU*mC*GACAAGAAAGGGACUGAGCCCUAGAGCG (SEQ ID |
| | NO: 10) |
| Dnr-03 | pGCGAAAGCCGCAAGUUAGGGUAAGGCUAGUCCGUUAUCAACUUGA |
| | AAAAGUGGGACCGAGUCGGUGCmU*mU*mU*U (SEQ ID NO: |
| | 11) |
| Acp-04 | mA*mU*mC*GACAAGAAAGGGACUGAGCCCUAGAGCCG (SEQ ID |
| | NO: 12) |
| Dnr-04 | pGCGGAAACGCCGGCAAGUUAGGGUAAGGCUAGUCCGUUAUCAACU |
| | UGAAAAAGUGGGACCGAGUCGGUGCmU*mU*mU*U (SEQ ID |
| | NO: 13) |
| Acp-05 | mA*mU*mC*GACAAGAAAGGGACUGAGUUUUAGAGCCG (SEQ ID |
| | NO: 14) |
| Dnr-05 | pGCGGAAACGCCGGCAAGUUAAAAUAAGGCUAGUCCGUUAUCAACU |
| | UGAAAAAGUGGGACCGAGUCGGUGCmU*mU*mU*U (SEQ ID |
| | NO: 15) |
| Acp-06 | mA*mU*mC*GACAAGAAAGGGACUGAGUUUUAGAGC (SEQ ID |
| | NO: 16) |
| Dnr-06 | pGCGGAAACGCGCAAGUUAAAAUAAGGCUAGUCCGUUAUCAACUUG |
| | AAAAAGUGGGACCGAGUCGGUGCmU*mU*mU*U (SEQ ID NO: |
| | 17) |
| Acp-07 | mA*mU*mC*GACAAGAAAGGGACUGAGUUUUAGAGC (SEQ ID |
| | NO: 18) |
| Dnr-07 | pUAGAAAUAGCAAGUUAAAAUAAGGCUAGUCCGUUAUCAACUUGAA |
| | AAAGUGGGACCGAGUCGGUGCmU*mU*mU*U (SEQ ID NO: |
| | 19) |
| Acp-08 | mA*mU*mC*GACAAGAAAGGGACUGAGUUUUAGAmGmC (SEQ ID |
| | NO: 20) |
| Dnr-08 | pmGmCmGmGmAmAmAmCmGmCmGmCAAGUUAAAAUAAGGCUAGUCC |
| | GUUAUCAmAmCmUmUmGmAmAmAmAmAmGmUmGmGmCmAmCmCmGm |
| | AmGmUmCmGmGmUmGmCmU*mU*mU*mU (SEQ ID NO: 21) |
| Acp-05_v2 | mG*mA*mA*CACAAAGCAUAGACUGCGUUUUAGAGCCG (SEQ ID |
| | NO: 22) |

mN indicates nucleotide with 2′OMe modification; N* indicates nucleotide with a 3′ phosphorothioate modification; “p” indicates position with a phosphate group.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method comprising, contacting a first RNA with a second RNA,
wherein the first RNA and the second RNA comprise at least five RNA nucleotides that are complementary, and wherein the contacting forms a stem structure or a stem loop structure, and
ligating the first RNA and the second RNA with a ligating enzyme
(i) within the stem structure, or
(ii) at an end of the stem structure, thereby forming a loop at the end of the stem structure.

2. The method of claim 1, wherein the contacting forms a stem structure and the ligating enzyme ligates the first RNA and the second RNA at an end of the stem structure, thereby forming a loop at the end of the stem structure.

3. The method of claim 1, wherein the contacting forms a stem loop structure and the ligating enzyme ligates the first RNA and the second RNA within a stem of the stem loop structure.

4. The method of any one of the preceding claims, wherein the ligating enzyme is selected from the group consisting of T4 RNA ligase 1, T4 RNA Ligase 2, RtcB Ligase, Thermo-stable 5′ App DNA/RNA Ligase, ElectroLigase, T4 DNA Ligase, T3 DNA Ligase, T7 DNA Ligase, Taq DNA Ligase, SplintR Ligase E. coli DNA Ligase, 9° N DNA Ligase, CircLigase, CircLigase II, DNA Ligase I, DNA Ligase III, and DNA Ligase IV.

5. The method of claim 2, wherein the ligating enzyme is T4 RNA ligase 1.

6. The method of claim 3, wherein the ligating enzyme is T4 RNA ligase 2.

7. The method of any one of the preceding claims, wherein the first and/or second RNA is chemically synthesized.

8. The method of any one of the preceding claims, wherein the first RNA is a clustered regularly interspersed short palindromic repeats (CRISPR) RNA (crRNA) and the second RNA is a trans-activating RNA (tracrRNA).

9. The method of any one of the preceding claims wherein a guide RNA (gRNA) is produced.

10. The method of any one of the preceding claims, wherein the first RNA and/or the second RNA is chemically synthesized.

11. The method of any one of claims 1-9, wherein the first and/or the second RNA is enzymatically synthesized.

12. The method of any one of the preceding claims, wherein ligating the first RNA and the second RNA with a ligating enzyme creates phosphodiester linkages between the first and the second RNA.

13. The method of any one of the preceding claims, wherein the first RNA and/or second RNA nucleotide is engineered to allow for non-covalent assembly.

14. The method of any one of the preceding claims, wherein the stem loop has a length of between about 2-50 nucleotides.

15. The method of any one of the preceding claims, wherein the first RNA and the second RNA comprise at least two RNA nucleotides that have perfect complementarity.

16. The method of claim 14, wherein the first RNA and the second RNA comprise at least three, four, fix, six or seven consecutive RNA nucleotides that have perfect complementarity.

17. The method of claim 15 or 16, wherein the RNA nucleotides that have perfect complementarity are present in a top stem and/or in a bottom stem.

18. The method of any one of the preceding claims, wherein the first RNA and the second RNA comprise at least five, six, or seven consecutive RNA nucleotides that are complementary at a lower stem formed by the first RNA and the second RNA.

19. The method of any one of the preceding claims, wherein the first RNA and the second RNA comprise at least four to fourteen consecutive RNA nucleotides that are complementary at an upper stem.

20. The method of claim 19, wherein the first RNA and the second RNA comprise four consecutive RNA nucleotides that are complementary at an upper stem.

21. The method of claim 19, wherein the first and the second RNA comprise five consecutive RNA nucleotides that are complementary at an upper stem.

22. The method of claim 19, wherein the first and the second RNA comprise seven consecutive RNA nucleotides that are complementary at an upper stem.

23. The method of claim 19, wherein the first and the second RNA comprises 14 consecutive RNA nucleotides that are complementary at an upper stem.

24. The method of any one of the preceding claims, wherein the first and the second RNA comprise 7 consecutive RNA nucleotides that are complementary at a lower stem.

25. The method of any one of the preceding claims, wherein the first RNA and/or the second RNA is engineered to create a ligation site for a ligation enzyme.

26. The method of any of the preceding claims, wherein the stem loop comprises a loop of 4, 5, 6, 7, 8, 9, 10, 11, 12, 13, 14, 15 or 16 nucleotides.

27. The method of any one of the preceding claims, wherein the stem loop comprises a tetraloop.

28. The method of claim 16, wherein the loop comprises 7 nucleotides.

29. The method of any one of claims 26-28, wherein ligating the first RNA and the second RNA occurs at a ligation site that is at least 1, 2, 3, 4, 5, 6, 7, 8, 9, or 10 base pairs from the loop.

30. The method of claim 29, wherein the ligation site is 2 or 3 base pairs from the loop.

31. The method of any one of the preceding claims, wherein ligating the first RNA and the second RNA occurs at a ligation site that is at least 3, 4, 5, 6, 7, 8, 9, 10, 11, or 12 base pairs from a bulge.

32. The method of claim 31, wherein ligating the first RNA and the second RNA occurs at a ligation site that is 3, 4, 5, or 11 base pairs from the bulge.

33. The method of any one of the preceding claims, wherein the first RNA and/or second RNA is enzymatically produced.

34. The method of any one the preceding claims, wherein the first RNA comprises a 3′ sequence that is capable of base pairing with a portion of the second RNA.

35. The method of any one of the preceding claims, wherein the first RNA comprises a phosphate at the 5′ terminus.

36. The method of claim 35, wherein the first RNA is a donor RNA.

37. The method of any one the preceding claims, wherein the second RNA comprises a variable protospacer region.

38. The method of claim 37, wherein the second RNA is an acceptor RNA.

39. The method of claim 35, wherein the first RNA comprises an adenosine triphosphate at the 5′ terminus.

40. The method of any one of the preceding claims, wherein about 8-50 nucleotides are complementary and allow for base pairing between the first and the second RNA.

41. The method of claim 40, wherein the 8-50 nucleotides are partially complementary.

42. The method of claim 41, wherein the 8-50 nucleotides are from about 50% to 99% complementary.

43. The method of claim 41, wherein the 8-50 nucleotides are perfectly complementary.

44. The method of any one of the preceding claims, wherein the first and the second RNA have different nucleotide lengths.

45. The method of claim 44, wherein the first RNA has from about 20-100 nucleotides.

46. The method of claim any one of the preceding claims, wherein the second RNA has from about 20-70 nucleotides.

47. The method of any one of the preceding claims, wherein base pairing occurs in a lower stem.

48. The method of claim 47, wherein 7 nucleotides are complementary in the lower stem and allow for base pairing between the first RNA and the second RNA.

49. The method of any claim 47 or 48, wherein the base pairing occurs in an upper stem.

50. The method of claim 49, wherein 2 nucleotides are complementary in the upper stem and allow for base pairing between the first RNA and the second RNA.

51. The method of any one of claims 9-50, wherein the gRNA has a length of about 100 nucleotides, about 125 nucleotides, about 150 nucleotides, about 175 nucleotides, about 200 nucleotides, or greater than about 200 nucleotides.

52. The method of any one of the preceding claims, wherein the gRNA is an extended guide RNA, prime editor guide RNA (pegRNA), or a Cas12 guide RNA such as Cas12a guide RNA, Cas12b guide RNA, Cas12c guide RNA, Cas12d, guide RNA, Cas12e guide RNA, Cas12f guide RNA, Cas12g guide RNA, Cas12h guide RNA, Cas12i guide RNA, Cas12j guide RNA, or Cas12k guide RNA.

53. The method of any one of the preceding claims, wherein the gRNA comprises one or more of the following: a spacer, a lower stem, a bulge, an upper stem, a nexus and a hairpin.

54. The method of any one of the preceding claims, wherein the first RNA and the second RNA are present at a ratio of about 0.5:1, 0.6:1, 0.7:1, 0.8:1, 0.9:1, 1:1, 1:0.9, 1:0.8, 1:0.7, 1:0.6, or 1:0.5.

55. The method of any one of the preceding claims, wherein the gRNA is produced at a yield of about 50%, 55%, 60%, 65%, 70%, 75%, 80%, 85%, 90%, 95%, 99% or more.

56. The method of any one of the preceding claims, wherein the gRNA is produced at 50%, 55%, 60%, 65%, 70%, 75%, 80%, 85%, 90%, 95%, 99% or more improvement in yield as compared to conventional synthetic methods.

57. A method of producing a synthetic guide RNA (gRNA) comprising:
providing a first RNA comprising a 5′-monophosphate;
providing a second RNA′;
providing an oligonucleotide that has partial complementarity to the first RNA and the second RNA, wherein the complementarity of the oligonucleotide allows for base pairing with the first and the second RNA; and
providing a ligase to catalyze ligation between the first and the second RNA, thus producing the synthetic gRNA.

58. A method of producing a synthetic guide RNA (gRNA) comprising:
providing a first RNA comprising a 5′-monophosphate;
providing a second RNA comprising a blocked 3′ end; and
providing a ligase to catalyze ligation between the first and the second RNA, thus producing the synthetic gRNA.

59. The method of any one of claims 57-58, wherein the first RNA is a trans-activating RNA (tracrRNA), and the second RNA is a clustered regularly interspersed short palindromic repeats (CRISPR) RNA (crRNA).

60. The method of claim 57, wherein the oligonucleotide is about 100 nucleotides long.

61. A method of producing a synthetic guide RNA (gRNA) comprising:
providing two or more RNA fragments;
providing an oligonucleotide that has partial complementarity to the two or more RNA fragments, wherein the complementarity of the oligonucleotide allows for base pairing with the two or more RNA fragments; and
providing a ligase to catalyze ligation between the two or more RNA fragments, thus producing the synthetic guide RNA.

62. The method of claim 61, wherein the two or more RNA fragments are ligated at an overhang, blunt end, or at a bulge.

63. A guide RNA (gRNA) or prime editing guide RNA (pegRNA) synthesized by the method of any one of claim 1-62.

64. A method for targeted transcription activation, targeted transcription repression, targeted epigenome modification, or targeted genome modification, the method comprising introducing into a eukaryotic cell:
(a) a synthetic guide RNA (gRNA) as defined in any one of the preceding claims;
(b) at least one CRISPR/Cas protein or a nucleic acid encoding the at least one CRISPR/Cas protein;
wherein interactions between (a) and (b) and a target sequence in chromosomal DNA leads to targeted transcription activation, targeted transcription repression, targeted epigenome modification, or targeted genome modification.

65. A method for targeted RNA modification, the method comprising introducing into a eukaryotic cell:
(a) a synthetic guide RNA (gRNA) as defined in any one of the preceding claims;
(b) at least one CRISPR/Cas protein or a nucleic acid encoding the at least one CRISPR/Cas protein;
wherein interactions between (a) and (b) and an RNA expressed by chromosomal DNA leads to a modification of the RNA expressed by the chromosomal DNA.

66. The method of claim 65, wherein the RNA expressed by the chromosomal DNA is a messenger RNA (mRNA).

67. The method of any one of claims 64-66, wherein the CRISPR/Cas protein is selected from Cas9, Cpf1, SaCas, Cas12, Cas13, or modified versions thereof.

68. A method for producing synthetic guide RNA (gRNA) according to any one of claims 1-67.

69. The method of claim 68, wherein the second RNA comprises a 3′ sequence that is capable of base pairing with a portion of the first RNA.

70. The method of claim 68 or 69, wherein the second RNA comprises a variable protospacer region.

71. The method of any one of claims 68-70, wherein the first RNA comprises a phosphate at the 5′ terminus.

72. The method of any one of claims 68-71, wherein the contacting forms a stem loop structure and the ligating enzyme ligates the first RNA and the second RNA within a stem of the stem loop structure.

73. The method of claim 72, wherein the ligating enzyme is T4 RNA ligase 2.

74. The method of claim 73, wherein the stem loop comprises GC base pairs in the upper stem.

75. The method of claim 74, wherein the upper stem comprises a nucleotide sequence at least about 80% identical to CGAUACGACAGAAC.

76. The method of claim 74, wherein the upper stem comprises a nucleotide sequence at least about 80% identical to CGCCG.

77. The method of claim 74, wherein the upper stem comprises a nucleotide sequence at least about 80% identical to CGGCCGC.

78. The method of claim 74, wherein the upper stem comprises a nucleotide sequence at least about 80% identical to CGCGC.

79. The method of claim 74, wherein the upper stem comprises a nucleotide sequence at least about 80% identical to CGAU.

80. The method of claim 72, wherein the stem loop comprises GC base pairs in the lower stem.

81. The method of any one of claims 68-79, wherein the lower stem does not comprise GC base pairs.

82. The method of any one of claims 68-81, wherein the upper stem does not comprise a GC base pair.

83. The method of any one of claims 68-81, wherein the upper the stem comprises at least 1, 2, 3, 4, 5, or 6, 7, 8, 9, 10, 11, or 12 GC base pairs.

84. The method of claim 83, wherein the upper portion of the stem comprises 2 GC nucleotides.

85. The method of any one of claims 68-84, wherein ligating the first and the second RNA results in a yield of at least 60%, 70%, 80%, 90%, or more than 95% of full-length product.

86. The method of claim 85, wherein ligating the first and the second RNA results in a yield of at least about 60%.

87. The method of any one of the claim 1-63 or 68-86, wherein the gRNA is produced at a quantity of at least 1 gram.

88. The method of claim 87, wherein large scale comprises at least 5 grams, 10 grams, 20 grams, 30 grams, 40 grams, 50 grams, 60 grams, 70 grams, 80 grams, 90 grams, or 100 grams.

89. The method of any one of claim 1-63 or 68-88, wherein the gRNA is produced at a quantity of less than 1 gram.

90. The method of claim 89, wherein the gRNA is produced at a quantity of about 0.05 grams, 0.1 grams, 0.2 grams, 0.3 grams, 0.4 grams, 0.5 grams, 0.6 grams, 0.7 grams, 0.8 grams, or 0.9g.

91. The method of any one of claims 68-90, wherein the method produces gRNA at a purity of about 50%, 60%, 70%, 80%, 90%, or more than 90%.

92. The method of any one of claims 68-91, wherein the first RNA is synthesized in a 3′ to 5′ direction.

93. The method of any one of claims 68-92, wherein the second RNA is synthesized in a 3′ to 5′ direction.

94. The method of any one of claims 68-93, wherein the gRNA has a length of about 100 nucleotides, about 125 nucleotides, about 150 nucleotides, about 175 nucleotides, about 200 nucleotides, or greater than about 200 nucleotides.

95. The method of any one of claims 68-94, wherein the loop comprises 4, 5, 6, 7, 8, 9, 10, 11, 12, 13, 14, 15 or 16 nucleotides.

96. The method of claim 95, wherein the loop is a tetraloop.

97. The method of claim 95, wherein the loop comprises 7 nucleotides.

98. The method of any one of claims 68-97, wherein ligating the first RNA and the second RNA occurs at a ligation site that is at least about 3 base pairs from the loop.

99. The method of claim 98, wherein the ligation site is 1, 2, 3, 4, 5, 6, or 10 base pairs from the loop.

100. The method of any one of claim 1-63 or 68-99, wherein the first and/or second RNA comprises one or more backbone modifications.

101. The method of claim 100, wherein the one or more backbone modifications comprises a 2′ O-methyl or a phosphorothioate modification.

102. The method of claim 100, wherein the one or more backbone modifications is selected from 2′-O-methyl 3′-phosphorothioate, 2′O-methyl, 2′-ribo 3′-phosphorothioate, deoxy, or 5′ phosphate modification.

103. The method of claim 101 or 102, wherein the one or more modifications are present at the site of ligation.

104. The method of claim 103, wherein the one or more modifications are present in the donor RNA and/or the acceptor RNA.

105. The method of claim 104, wherein the 3′ and/or the 5′ end of the donor RNA has one or more backbone modifications.

106. The method of claim 104, wherein the 3′ and/or the 5′ end of the acceptor RNA has one or more backbone modifications.

107. The method of any one of claim 1-63 or 68-106, wherein the concentration of the first and/or second RNA is between about 1 g/L and 5 g/L.

108. The method of claim 107, wherein the concentration of the first and/or second RNA is about 1 g/L.

109. The method of claim 107, wherein the concentration of the first and/or second RNA is about 3 g/L.

110. A composition produced by the method of any one of the proceeding claims comprising a first RNA comprising a phosphate at a 5′ terminus and a second RNA comprising a variable protospacer region, wherein the first and the second RNA are non-covalently bound.

111. A composition produced by the method of any one of claims 1-103 comprising a first RNA comprising a phosphate at a 5′ terminus and a second RNA comprising a variable protospacer region, and wherein the first and the second RNA are bound to a ligase.

112. The composition of claim 111, wherein the ligase is a T4 RNA ligase 2.

113. A composition comprising an RNA comprising a nucleotide sequence at least about 80% identical to CGAUACGACAGAAC.

114. The composition of claim 113, wherein the nucleotide sequence is identical to CGAUACGACAGAAC.

115. A composition comprising an RNA comprising a nucleotide sequence at least about 80% identical to CGCCG.

116. The composition of claim 115, wherein the nucleotide sequence is identical to CGCCG.

117. A composition comprising an RNA comprising a nucleotide sequence at least about 80% identical to CGGCCGC.

118. The composition of claim 117, wherein the nucleotide sequence is identical to CGGCCGC.

119. A composition comprising an RNA comprising a nucleotide sequence at least about 80% identical to CGCGC.

120. The composition of claim 119, wherein the nucleotide sequence is identical to CGCGC.

121. A kit comprising the composition of any one of claims 110-120.

122. A kit comprising a first RNA comprising trans-activating RNA (tracrRNA) sequence, a second RNA comprising a variable protospacer region, and a ligase.

123. The kit of claim 122, wherein the ligase is a T4 RNA Ligase 2.
